(19) United States
(12) Patent Application Publication
(10) Pub. No.: US 2005/0226927 A1
(43) Pub. Date: Oct. 13, 2005

(54) Pharmaceutical dosage forms having immediate release and/or controlled release properties that contain a GABAB receptor agonist

(75) Inventors: Chien-Hsuan Han, Sunnyvale, CA (US); Ann F. Hsu, Los Altos Hills, CA (US); Larry Hsu, Los Altos Hills, CA (US); Charles Hsiao, Pleasanton, CA (US); Ching-Ling Diana Teng, Fremont, CA (US)

Correspondence Address: S2IPLAW, PLLC, 300 MASSACHUSETTS AVENUE, NW, SUITE 1101, WASHINGTON, DC, 20001-2692, US

(73) Assignee: Impax Laboratories, Inc.

(21) Appl. No.: 11/138,008

(22) Filed: May 26, 2005

Related U.S. Application Data
(63) Continuation-in-part of application No. 10/815,924, filed on Apr. 2, 2004.
(63) Continuation-in-part of application No. 10/815,926, filed on Apr. 2, 2004.
(63) Continuation-in-part of application No. 10/815,929, filed on Apr. 2, 2004.
(63) Continuation-in-part of application No. 10/815,930, filed on Apr. 2, 2004.

(51) Int. Cl. A61K 31/195; A61K 9/22

(52) U.S. Cl. 424/468; 514/561

(57) ABSTRACT

The present invention relates generally to pharmaceutical dosage forms having immediate release and controlled release properties that contain a γ-aminobutyric acid (GABA_{B}) receptor agonist, e.g., baclofen, for the treatment of medical conditions, which includes spasms, cramping, and tightness of muscles, associated with ailments such as multiple sclerosis or certain spinal injuries.

### CROSS-REFERENCE TO RELATED APPLICATIONS

This application is a continuation-in-part, and claims the benefit under 35 USC 120 of, U.S. patent application Ser. No. 10/815,924 filed 2 Apr. 2004; U.S. patent application Ser. No. 10/815,926 filed 2 Apr. 2004; U.S. patent application Ser. No. 10/815,929 filed 2 Apr. 2004; and U.S. patent application Ser. No. 10/815,930 filed 2 Apr. 2004; which are incorporated herein by reference.

### BACKGROUND OF THE INVENTION

The present invention relates generally to pharmaceutical dosage forms having immediate release and controlled release properties that contain a γ-aminobutyric acid (GABA_{B}) receptor agonist, e.g., baclofen, for the treatment of medical conditions, which include spasms, cramping, tightness of muscles, or spasticity associated with ailments such as multiple sclerosis, spinal cord diseases or certain spinal injuries.

Multiple sclerosis is considered to be an autoimmune disease. In this regard, an individual's immune system can attack the myelin sheath that surrounds nerve cells. This damage leads to muscle weakness, paralysis, poor coordination, balance problems, fatigue, and possible blindness. The GABA_{B} agonist baclofen can be used to treat these symptoms. Baclofen can also facilitate adjunct medical treatment, such as physical therapy, to improve the condition of a patient with multiple sclerosis or certain spinal injuries. Baclofen is also used to reduce the number and severity of attacks of trigeminal neuralgia in patients who are not able to tolerate, or who have become refractory to, the effects of carbamazepine.

Baclofen, or 4-amino-3-(4-chlorophenyl)-butanoic acid, is a muscle relaxant and antispastic. Its mechanism of action appears unclear. Baclofen seems capable of inhibiting both monosynaptic and polysynaptic reflexes at the spinal level, possibly by hyperpolarization of afferent terminals, although actions at supraspinal sites may also occur and contribute to its clinical effect. In studies with animals, baclofen has been shown to have general central nervous system (CNS) depressant properties as indicated by the production of sedation with tolerance, somnolence, ataxia, and respiratory and cardiovascular depression.

The absorption of baclofen is site specific. Baclofen is primarily absorbed in the upper gastrointestinal (GI) tract, with the extent of absorption of baclofen substantially reduced in the lower GI tract. Baclofen is rapidly and extensively absorbed. Absorption may be dose-dependent, being reduced with increasing doses. An improved method of administering baclofen to a patient would include the delivery of effective amounts of the drug to the upper GI tract for an extended period.

Several side effects are possibly associated with the administration of baclofen to mammals. These problems include nausea, vomiting, diarrhea, dizziness, daytime sedation, and less frequently, psychotic states such as depressive mood disorder. In addition, patient compliance with a dosing regimen can be suboptimal where frequent doses are required, such as the need for administering a dosage form three or four times a day. A pharmaceutical dosage form that requires less frequent dosing, such as once or twice a day, thus would be preferable. Furthermore, a pharmaceutical dosage form capable of establishing and maintaining stable plasma levels of baclofen for a prolonged period of time may benefit patients by requiring less frequent dosing and by minimizing side effects.

Certain baclofen pharmaceutical formulations, including Baclofen Tablet, 10/20 mg (Watson Pharmaceuticals, Inc., Corona, Calif.) and the orally disintegrating tablet marketed as KEMSTRO™ (Schwarz Pharma, Monheim, Germany), are marketed commercially, but do not provide controlled release of baclofen. For example, following a single 20 mg oral dose of KEMSTRO™, the peak plasma concentration is reached about 1½ hours after administration.

Baclofen has a serum half-life of 2.5 to 4 hours. (Drug Monograph: Baclofen American Hospital Formulary Service (AHFS) American Society of Hospital Pharmacists, Inc., Bethesda, Md. 2003). Following oral administration of existing immediate release baclofen formulations, minimum therapeutic plasma levels are typically reached at about four to eight hours following administration. Therefore, existing immediate-release formulations typically require dosing three to four times daily.

Various controlled release baclofen compositions have been reported. For example, U.S. Pat. No. 5,091,184, issued Feb. 25, 1992, to Khanna describes adhesive tablets that stick to the oral mucosa and deliver drug through the mucous membrane. These compositions have one or more of the problems associated with adhesive tablets and deliver the drug to a less than optimal site for GABA related drugs. Additionally, U.S. Pat. No. 5,651,985, issued Jul. 29, 1997, to Penners et al. refers to matrix dosage forms having extended gastric residence time. Dosage forms made according to the Penners reference are described as having marked swelling and high dimensional stability in the swollen state. In addition, an osmotic pump type dosage form for delivering baclofen is referred to in U.S. Pat. No. 4,764,380, issued Aug. 16, 1988, to Urquhart et al., which describes the continuous administration of drug over a prolonged period of time.

Nevertheless, there remains a significant and continuing need for pharmaceutical dosage forms having controlled release properties that contain a GABA_{B} receptor agonist, such as baclofen, to treat medical conditions like multiple sclerosis or certain spinal injuries by establishing and maintaining stable plasma levels of the drug for a prolonged period of time to achieve less frequent dosing and to minimize side effects. These and other objectives are accomplished by the present invention.

### SUMMARY OF THE INVENTION

The present invention relates generally to pharmaceutical dosage forms having controlled release properties that contain a GABA_{B} receptor agonist, such as baclofen. These dosage forms can be used in the treatment of medical conditions, like spasms, cramping, and tightness of muscles, which are associated with ailments such as multiple sclerosis or certain spinal injuries.

For example, the pharmaceutical dosage forms of the present invention may involve a controlled release dosage form, where the controlled release dosage form includes a GABA_{B} agonist and a pharmaceutically acceptable excipient, and the dosage form exhibits an in vitro dissolution profile in simulated intestinal fluid medium comprising less than about 70% GABA_{B} agonist release after 1 hour, at least about 20% GABA_{B} agonist release after 4 hours, and at least about 30% GABA_{B} agonist release after 6 hours. In this embodiment, the controlled release dosage form exhibits an in vitro dissolution profile in simulated gastric fluid/simulated intestinal fluid (1 hour switchover) medium comprising less than about 80% GABA_{B} agonist release after 1 hour, at least about 30% GABA_{B} agonist release after 4 hours, and at least about 40% GABA_{B} agonist release after 6 hours.

In a preferred embodiment, the controlled release dosage form including the GABA_{B} agonist and pharmaceutically acceptable excipient exhibits an in vitro dissolution profile in simulated intestinal fluid medium comprising less than about 50% GABA_{B} agonist release after 1 hour, at least about 40% GABA_{B} agonist release after 4 hours, and at least about 50% GABA_{B} agonist release after 6 hours. In this preferred embodiment, the controlled release dosage form exhibits an in vitro dissolution profile in simulated gastric fluid/simulated intestinal fluid (1 hour switchover) medium comprising less than about 70% GABA_{B} agonist release after 1 hour, at least about 40% GABA_{B} agonist release after 4 hours, and at least about 50% GABA_{B} agonist release after 6 hours

In another embodiment, the controlled-release GABA_{B} agonist dosage form is combined with an immediate release GABA_{B} agonist component. In this embodiment, the immediate release component exhibits an in vitro dissolution profile in simulated gastric fluid comprising at least about 80% GABA_{B} agonist release after 1 hour. The ratio of the immediate-release component to the controlled-release component will be from about 1:10 to about 10: 1, preferably from about 1:4 to about 4:1, more preferably from about 1:3 to about 3:1, and most preferably from about 1:2 to about 2:1.

In another embodiment, the pharmaceutical dosage forms of the present invention contain an enteric-coated controlled release component, where the enteric-coated controlled release component includes a GABA_{B} agonist and a pharmaceutically acceptable excipient, and the enteric-coated controlled release component exhibits an in vitro dissolution profile in simulated gastric fluid/simulated intestinal fluid (2 hour switchover) medium comprising less than about 10% GABA_{B} agonist release after 2 hours, at least about 40% GABA_{B} agonist release after 3 hours, and at least about 70% GABA_{B} agonist release after 6 hours. Preferably, the enteric-coated controlled release component exhibits an in vitro dissolution profile in simulated gastric fluid/simulated intestinal fluid (2 hour switchover) medium comprising less than about 10% GABA_{B} agonist release after 2 hours, at least about 50% GABA_{B} agonist release after 3 hours, and at least about 80% GABA_{B} agonist release after 6. Most preferably, the enteric-coated controlled release component exhibits an in vitro dissolution profile in simulated gastric fluid/simulated intestinal fluid (2 hour switchover) medium comprising less than about 10% GABA_{B} agonist release after 2 hours, at least about 60% GABA_{B} agonist release after 3 hours, and at least about 90% GABA_{B} agonist release after 6 hours.

In a further preferred embodiment, the dosage form also contains an immediate release component, in combination with the enteric-coated controlled release component. For example, the GABA_{B} agonist may be formulated as a combination of immediate-release beads and controlled-release beads, compressed into a tablet or contained in a capsule dosage form. The ratio of the immediate-release component to the controlled-release component will be from about 1:10 to about 10:1, preferably from about 1:4 to about 4:1, more preferably from about 1:3 to about 3:1, and most preferably from about 1:2 to about 2:1.

The present invention includes pharmaceutical dosage forms having both immediate release and extended release properties. In this embodiment, the pharmaceutical dosage form comprising a GABA_{B} agonist and a pharmaceutically acceptable excipient exhibits an in vitro dissolution profile in simulated gastric fluid/simulated intestinal fluid (2 hour switchover) medium comprising less than about 75% GABA_{B} agonist release after 2 hours, and at least about 80% GABA_{B} agonist release after 3 hours. Preferably, the pharmaceutical dosage form exhibits an in vitro dissolution profile in simulated gastric fluid/simulated intestinal fluid (2 hour switchover) medium comprising less than about 65% GABA_{B} agonist release after 2 hours, and at least about 90% GABA_{B} agonist release after 3 hours

The pharmaceutical dosage forms of the present invention are adapted to be administered twice daily in patients requiring chronic baclofen therapy. As such, the in vivo absorption is prolonged as compared to immediate release baclofen formulations, such that the median time period at which at least 80% of said baclofen is absorbed, in vivo, under fasting conditions, is greater than 2.5 hours. The dosage forms of the present invention will typically exhibit an in vivo plasma profile comprising mean maximum baclofen levels (Cₘₐₓ) from about 30 minutes to about 7 hours after administration to a fasting patient, often between 2.5 and 5.5 hours after administration.

### BRIEF DESCRIPTION OF THE DRAWINGS

FIG. 1A is a graph of the in vitro dissolution profile of a baclofen tablet formulation, 20 mg, prepared according to Example 8, according to measurements under the USP paddle method of 50 rpm in 900 ml simulated gastric fluid (pH 1.2) at 37° C.

FIG. 1B is a graph of the in vitro dissolution profile of a baclofen tablet formulation, 20 mg, prepared according to Example 11, according to measurements under the USP paddle method of 50 rpm in 900 ml simulated intestinal fluid (pH 6.8) at 37° C.

FIG. 2 is a graph of the in vitro dissolution profiles of baclofen tablet formulations, 20 mg, prepared according to Example 11, according to measurements under the USP paddle method of 50 rpm in 900 ml simulated gastric fluid (pH 1.2) at 37° C. for 1 hour with a switchover to simulated intestinal fluid (pH 6.8).

FIG. 3 is a graph of the in vitro dissolution profile of a baclofen capsule formulation, 20 mg, prepared according to Example 2, according to measurements under the USP paddle method of 75 rpm in 900 ml simulated gastric fluid (pH 1.2) at 37° C.

FIG. 4 is a graph of the in vitro dissolution profiles of baclofen capsule formulations, 20 mg, prepared according to Example 12, according to measurements under the USP paddle method of 75 rpm in 900 ml simulated gastric fluid (pH 1.2) at 37° C. for 2 hours with a switchover to simulated intestinal fluid (pH 6.8).

FIG. 5 is a graph of the in vitro dissolution profiles of baclofen capsule formulations, 30 mg, prepared according to Examples 13 and 14, according to measurements under the USP paddle method of 75 rpm in 900 ml simulated gastric fluid (pH 1.2) at 37° C. for 2 hours with a switchover to simulated intestinal fluid (pH 6.8).

FIG. 6 is a graph of the in vivo plasma profiles of baclofen tablet formulations, determined as described in Example 16.

FIG. 7 is a graph simulating steady-state baclofen plasma levels determined as described in Example 17, where (C) represents the 40 mg dosage form of the present invention and (D) represents the reference 20 mg immediate-release dosage form.

### DETAILED DESCRIPTION OF THE INVENTION

The present invention relates to pharmaceutical dosage forms comprising a controlled-release GABA_{B} agonist (preferably baclofen, a baclofen prodrug, a baclofen analog, or a mixture thereof, as well as a racemic baclofen mixture or a substantially pure L-baclofen enantiomeric product) formulation. The controlled-release GABA_{B} agonist formulation may be in the form of an enteric-coated controlled-release formulation. In addition, the controlled-release GABA_{B} agonist formulations, including enteric-coated controlled-release formulations, may be combined with immediate release GABA_{B} agonist formulations in the final pharmaceutical dosage forms.

It has been found that the formulations of the present invention allow for less frequent dosing as compare to existing immediate release formulations. For example, for patients requiring chronic GABA_{B} agonist therapy, twice daily administration of the formulations of the present invention is bioequivalent to three times daily administration of an existing immediate release formulation. This reduced dosing frequency is more convenient for patients, and typically leads to better patient compliance. In addition, it reduces the number of plasma peaks and troughs, which is typically associated with improved efficacy and reduced side effects.

As used herein and in the claims, the singular forms “a,” “an,” and “the” include the plural reference unless the context clearly indicates otherwise. Thus, for example, the reference to a profile is a reference to one or more such profiles, including equivalents thereof known to those skilled in the art. Other than in the operating examples, or where otherwise indicated, all numbers expressing quantities of ingredients or reaction conditions used herein should be understood as modified in all instances by the term “about.” The term “about” when used in connection with percentages can mean±1%.

All patents and other publications identified are incorporated herein by reference for the purpose of describing and disclosing, for example, the methodologies described in such publications that might be used in connection with the present invention.

Unless defined otherwise, all technical and scientific terms used herein have the same meaning as those commonly understood to one of ordinary skill in the art to which this invention pertains. Although any known methods, devices, and materials may be used in the practice or testing of the invention, the preferred methods, devices, and materials in this regard are described here.

The baclofen, also known as butanoic acid or 4-amino-3-(4-chlorophenyl)butanoic acid, of the present invention includes racemic baclofen, enantiomerically pure L-baclofen, and analogs, derivatives, prodrugs, metabolites thereof, and any pharmaceutically acceptable salts thereof.

Baclofen is a GABA_{B} receptor agonist, and thus other GABA_{B} receptor agonists are envisioned within the scope of the invention. These may include 4-aminobutanoic acid (GABA); 3-aminopropyl)methylphosphinic acid; 4-amino-3-phenylbutanoic acid; 4-amino-3-hydroxybutanoic acid; 4-amino-3-(4-chlorophenyl)-3-hydroxyphenylbutanoic acid; 4-amino-3-(thien-2-yl)butanoic acid; 4-amino-3-(5-chlorothien-2-yl)butanoic acid; 4-amino-3-(5-bromothien-2-yl)butanoic acid; 4-amino-3-(5-methylthien-2-yl)butanoic acid; 4-amino-3-(2-imidazolyl)butanoic acid; 4-guanidino-3-(4-chlorophenyl)butanoic acid; 3-amino-2-(4-chlorophenyl)-1-nitropropane; (3-aminopropyl)phosphonous acid; (4-aminobut-2-yl)phosphonous acid; (3-amino-2-methylpropyl)phosphonous acid; (3-aminobutyl)phosphonous acid; (3-amino-2-(4-chlorophenyl)propyl)phosphonous acid; (3-amino-2-(4-chlorophenyl)-2-hydroxypropyl)phosphonous acid; (3-amino-2-(4-fluorophenyl)propyl)phosphonous acid; (3-amino-2-phenylpropyl)phosphonous acid; (3-amino-2-hydroxypropyl)phosphonous acid; (E)-(3-aminopropen-1-yl)phosphonous acid; (3-amino-2-cyclohexylpropyl)phosphonous acid; (3-amino-2-benzylpropyl)phosphonous acid; [3-amino-2-(4-methylphenyl)propyl]phosphonous acid; [3-amino-2-(4-trifluoromethylphenyl)propyl]phosphonous acid; [3-amino-2-(4-methoxyphenyl)propyl]phosphonous acid; [3-amino-2-(4-chlorophenyl)-2-hydroxypropyl]phosphonous acid; (3-amino propyl)methylphosphinic acid; (3-amino-2-hydroxypropyl)methylphosphinic acid; (3-aminopropyl)(difluoromethyl)phosphinic acid; (4-aminobut-2-yl)methylphosphinic acid; (3-amino-1-hydroxypropyl)methylphosphinic acid; (3-amino-2-hydroxypropyl)(difluoromethyl)phosphinic acid; (E)-(3-aminopropen-1-yl)methylphosphinic acid; (3-amino-2-oxo-propyl)methyl phosphinic acid; (3-aminopropyl)hydroxymethylphosphinic acid; (5-aminopent-3-yl)methylphosphinic acid; (4-amino-1,1,1-trifluorobut-2-yl)methylphosphinic acid; (3-amino-2-(4-chlorophenyl)propyl)sulfinic acid; 3-aminopropylsulfinic acid, 1-(aminomethyl)cyclohexaneacetic acid, and the like. See, e.g., U.S. Pat. No. 6,664,069.

The term “analog” means a compound which comprises a chemically modified form of a specific compound or class thereof, and which maintains the pharmaceutical and/or pharmacological activities characteristic of said compound or class. For example, baclofen analogs include 3-thienyl- and 3-furylaminobutyric acids.

The term “derivative” means a chemically modified compound wherein the modification is considered routine by the ordinary skilled chemist, such as an ester or an amide of an acid, protecting groups, such as a benzyl group for an alcohol or thiol, and tert-butoxycarbonyl group for an amine.

The term “prodrug”, as used herein, includes any covalently bonded carriers which release an active parent drug of the present invention in vivo when such prodrug is administered to a patient. Because prodrugs are known to enhance numerous desirable qualities of pharmaceuticals (i.e., solubility, bioavailability, manufacturing, etc.) the compounds of the present invention may be delivered in prodrug form. Prodrugs of the present invention may be prepared by modifying functional groups present in the compound in such a way that the modifications are cleaved, either in routine manipulation or in vivo, to the parent compound. The transformation in vivo may be, for example, as the result of some metabolic process, such as chemical or enzymatic hydrolysis of a carboxylic, phosphoric or sulphate ester, or reduction or oxidation of a susceptible functionality. Prodrugs within the scope of the present invention include compounds wherein a hydroxy, amino, or sulfhydryl group is bonded to any group that, when the prodrug of the present invention is administered to a mammalian subject, it cleaves to form a free hydroxyl, free amino, or free sulfhydryl group, respectively. Functional groups that may be rapidly transformed, by metabolic cleavage, in vivo form a class of groups reactive with the carboxyl group of the compounds of this invention. They include, but are not limited to, such groups as alkanoyl (such as acetyl, propionyl, butyryl, and the like), unsubstituted and substituted aroyl (such as benzoyl and substituted benzoyl), alkoxycarbonyl (such as ethoxycarbonyl), trialkysilyl (such as trimethyl- and triethysilyl), monoesters formed with dicarboxylic acids (such as succinyl), and the like. Because of the ease with which metabolically cleavable groups of the compounds useful according to this invention are cleaved in vivo, the compounds bearing such groups act as prodrugs. The compounds bearing the metabolically cleavable groups have the advantage that they may exhibit improved bioavailability as a result of enhanced solubility and/or rate of absorption conferred upon the parent compound by virtue of the presence of the metabolically cleavable group.

A discussion of prodrugs is provided in the following: DESIGN OF PRODRUGS, H. Bundgaard, ed. (Elsevier, 1985); METHODS IN ENZYMOLOGY, K Widder et al., eds., vol. 42, 309-96 (Academic Press 1985); A TEXTBOOK OF DRUG DESIGN AND DEVELOPMENT, Krogsgaard-Larsen & H. Bundgaard, ed., Chapter 5; Design and Applications of Prodrugs, 113-91 (1991); H. Bundgard, Advanced Drug Delivery Reviews, 1-38 (1992); 8 J. PHARM. SCIENCES 285 (1988); N. Nakeya et al., 32 CHEM. PHARM. BULL. 692 (1984); T. Higuchi and V. Stella, Prodrugs as Novel Delivery Systems, 14 A.C.S. SYMPOSIUM SERIES: BIOREVERSIBLE CARRIERS IN DRUG DESIGN, Edward B. Roche, ed. (Am. Pharm. Assoc. & Pergamon Press 1987), each of which is incorporated herein by reference.

Thus, the present invention contemplates the use of prodrugs of GABA_{B} receptor agonists (including baclofen), methods of delivering the same, and compositions containing the same. For example, baclofen prodrugs have been described in Leisen et al., Lipophilicities of Baclofen Ester Prodrugs Correlate with Affinities to the ATP-dependent Efflux Pump P-glycoprotein, 20 PHARM. RES. 772-78 (2003).

The term “metabolite” refers to a form of a compound obtained in a human or animal body by action of the body on the administered form of the compound, for example a de-methylated analog of a compound bearing a methyl group which is obtained in the body after administration of the methylated compound as a result of action by the body on the methylated compound. Metabolites may themselves have biological activity.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

For example, “pharmaceutically acceptable salts” refer to derivatives of the disclosed compounds wherein the specified compound is converted to an acid or base salt thereof Such pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluensulfonic, methanesulfonic, ethane dislfonic, oxalic, isethionic, and the like.

For purposes of the present invention, the term “controlled release” refers to part or all of a dosage form that can release one or more active pharmaceutical agents over a prolonged period of time (i.e., over a period of more than 1 hour), or delays the release of active agent for a prolonged period of time. The characteristic of controlled release (CR) may also be referred to as sustained release (SR), prolonged release (PR), modified release (MR), delayed release (DR) or extended release (ER). When used in association with the dissolution profiles discussed herein, the term “controlled release” refers to that portion of a dosage form according to the present invention that delivers active agent over a period of time greater than 1 hour.

“Immediate release” refers to part or all of a dosage form that releases active agent substantially immediately upon contact with gastric juices and that results in substantially complete dissolution within about 1 hour. The characteristic of immediate release (IR) may also be referred to as instant release (IR). When used in association with the dissolution profiles discussed herein, the term “immediate release” refers to that portion of a dosage form according to the present invention that delivers active agent over a period of time less than 1 hour.

The term “C_{MAX}” is the peak blood plasma concentration exhibited by the compositions of the present invention. “T_{MAX}” refers to the time that C_{MAX} occurs in the plasma concentration-time profile. “C_{MIN}” is the minimum plasma concentration. “C” is shorthand for concentration, “T” for time, “max” for maximum, and “min” for minimum. Initial peak plasma level refers to the first rise in blood plasma level of the active agent and may be followed by one or more additional peaks, one of which may be C_{MAX}. As used herein, “mean maximum GABA_{B} agonist level” refers to the mean GABA_{B} agonist C_{MAX}. The blood plasma concentrations described herein are typically determined across a population of at least 12 subjects.

The blood plasma concentrations described above may refer to plasma levels after a single oral administration of the dosage form, or may refer to levels obtained at steady state. As used herein, “steady state” blood plasma concentrations refers to the plasma levels obtained upon the repeated dosing of a drug until it reaches a stable level of absorption and elimination such that the amount of drug in the body is substantially constant.

As used herein, the term “patient” means any mammal including humans.

The term “effective amount” means an amount of a compound/composition according to the present invention effective in producing the desired therapeutic effect.

The term “excipients” refer to pharmacologically inert ingredients that are not active in the body. See HANDBOOK OF PHARMACEUTICAL EXCIPIENTS (Am. Pharm. Ass'n 1986). The artisan of ordinary skill in the art will recognize that many different excipients can be used in formulations according to the present invention and the list provided herein is not exhaustive.

The active ingredients of the present invention may be mixed with pharmaceutically acceptable carriers, diluents, adjuvants, excipients, or vehicles, such as preserving agents, fillers, polymers, disintegrating agents, glidants, wetting agents, emulsifying agents, suspending agents, sweetening agents, flavoring agents, perfuming agents, lubricating agents, acidifying agents, and dispensing agents, depending on the nature of the mode of administration and dosage forms. Such ingredients, including pharmaceutically acceptable carriers and excipients, may be used to formulate oral dosage forms. Pharmaceutically acceptable carriers include water, ethanol, polyols, vegetable oils, fats, waxes polymers, including gel forming and non-gel forming polymers, and suitable mixtures thereof Examples of excipients include starch, pregelatinized starch, Avicel, lactose, milk sugar, sodium citrate, calcium carbonate, dicalcium phosphate, and lake blend. Examples of disintegrating agents include starch, alginic acids, and certain complex silicates. Examples of lubricants include magnesium stearate, sodium lauryl sulphate, talc, as well as high molecular weight polyethylene glycols.

“Dosing under fasting conditions” is defined as when the dosage is administered orally with 240 ml of room temperature water after subjects are fasted overnight for at least 10 hours. No fluid, except that given with drug administration, will be allowed from 1 hour prior to dose administration until 1 hour after dosing. At 2 hours post-dose, subjects may consume 240 ml of room temperature water.

The pharmaceutical dosage forms of the present invention may involve a controlled release dosage form, where the controlled release dosage form includes a GABA_{B} agonist and a pharmaceutically acceptable excipient, and the dosage form exhibits an in vitro dissolution profile in simulated intestinal fluid medium comprising less than about 70% GABA_{B} agonist release after 1 hour, at least about 20% GABA_{B} agonist release after 4 hours, and at least about 30% GABA_{B} agonist release after 6 hours. In this embodiment, the controlled release dosage form exhibits an in vitro dissolution profile in simulated gastric fluid/simulated intestinal fluid (1 hour switchover) medium comprising less than about 80% GABA_{B} agonist release after 1 hour, at least about 30% GABA_{B} agonist release after 4 hours, and at least about 40% GABA_{B} agonist release after 6 hours.

Preferably, the controlled release dosage form exhibits an in vitro dissolution profile in simulated intestinal fluid medium comprising less than about 50% GABA_{B} agonist release after 1 hour, at least about 40% GABA_{B} agonist release after 4 hours, and at least about 50% GABA_{B} agonist release after 6 hours. In this preferred embodiment, the controlled release dosage form exhibits an in vitro dissolution profile in simulated gastric fluid/simulated intestinal fluid (1 hour switchover) medium comprising less than about 70% GABA_{B} agonist release after 1 hour, at least about 40% GABA_{B} agonist release after 4 hours, and at least about 50% GABA_{B} agonist release after 6 hours

In another embodiment, the controlled-release GABA_{B} agonist dosage form is combined with an immediate release GABA_{B} agonist component. In this embodiment, the immediate release component exhibits an in vitro dissolution profile in simulated gastric fluid comprising at least about 80% GABA_{B} agonist release after 1 hour.

In another embodiment, the pharmaceutical dosage forms of the present invention contain an enteric-coated controlled release component, where the enteric-coated controlled release component includes a GABA_{B} agonist and a pharmaceutically acceptable excipient, and the enteric-coated controlled release component exhibits an in vitro dissolution profile in simulated gastric fluid/simulated intestinal fluid (2 hour switchover) medium comprising less than about 10% GABA_{B} agonist release after 2 hours, at least about 40% GABA_{B} agonist release after 3 hours, and at least about 70% GABA_{B} agonist release after 6 hours. Preferably, the enteric-coated controlled release component exhibits an in vitro dissolution profile in simulated gastric fluid/simulated intestinal fluid (2 hour switchover) medium comprising less than about 10% GABA_{B} agonist release after 2 hours, at least about 50% GABA_{B} agonist release after 3 hours, and at least about 80% GABA_{B} agonist release after 6. Most preferably, the enteric-coated controlled release component exhibits an in vitro dissolution profile in simulated gastric fluid/simulated intestinal fluid (2 hour switchover) medium comprising less than about 10% GABA_{B} agonist release after 2 hours, at least about 60% GABA_{B} agonist release after 3 hours, and at least about 90% GABA_{B} agonist release after 6 hours.

In a further preferred embodiment, the dosage form also contains an immediate release component, in combination with the enteric-coated controlled release component.

The present invention includes pharmaceutical dosage forms having both immediate release and extended release properties. In this embodiment, the pharmaceutical dosage form comprising a GABA_{B} agonist and a pharmaceutically acceptable excipient exhibits an in vitro dissolution profile in simulated gastric fluid/simulated intestinal fluid (2 hour switchover) medium comprising less than about 75% GABA_{B} agonist release after 2 hours, and at least about 80% GABA_{B} agonist release after 3 hours. Preferably, the pharmaceutical dosage form exhibits an in vitro dissolution profile in simulated gastric fluid/simulated intestinal fluid (2 hour switchover) medium comprising less than about 65% GABA_{B} agonist release after 2 hours, and at least about 90% GABA_{B} agonist release after 3 hours

Appropriate in vitro dissolution testing methods for the dosage forms of the present invention are known to those of skill in the art and include those described in the Examples herein. The USP paddle method refers to the Paddle and Basket Method as described in United States Pharmacopoeia, Edition XXII (1990). In particular, the USP paddle method of 50 rpm or 75 rpm in 900 ml simulated gastric fluid (SGF) (pH 1.2) or simulated intestinal fluid (SIF) (pH 6.8) at 37° C. may be used to determine the in vitro dissolution profiles according to the present invention.

When the dosage forms of the present invention include a controlled release component, including enteric-coated controlled release, as well as an immediate release component, the ratio of the immediate release component to the controlled release component is from about 1:10 to about 10:1, preferably about 1:4 to about 4:1, more preferably from about 1:3 to about 3:1, and most preferably from about 1:2 to about 2:1.

The pharmaceutical dosage forms of the present invention are adapted to allow prolonged absorption of the active agent, which allows less frequent administration as compared to existing immediate-release formulations. As used herein, “prolonged absorption” means that the active agent is absorbed in vivo, under fasting conditions, over an extended period of time. In particular, the time period over which the majority (i.e., 80-90%) of the absorption occurs extends to about 7 or 8 hours after administration of the dosage form. Specifically, the median time period at which at least 80% of the active agent is absorbed from the dosage forms of the present invention is greater than 2.5 hours after administration, typically three to 4.5 hours after administration. By comparison, the median time period at which at least 80% of the active agent is absorbed from existing immediate-release formulations is 1.5 to two hours after administration. The period over which an active agent is absorbed from a dosage form can be calculated by deconvolution, using mathematical methods known to those of skill in the art.

The dosage forms of the present invention will exhibit an in vivo plasma profile comprising mean maximum GABA_{B} agonist levels from about 30 minutes to about 7 hours, often from about 2.5 hours to about 5.5 hours, after administration of a single dose to a fasting patient. At steady-state, the pharmaceutical dosage forms of the present invention will reach a C_{MIN} comparable to that obtained at steady-state from an immediate-release dosage form at a later time point, which will allow less frequent dosing. In particular, a 40 mg dosage form of the present invention, when administered twice daily, will deliver mean steady-state area under the plasma concentration-time curve (AUC), maximum plasma concentration (C_{MAX}), and minimum plasma concentration (C_{MIN}) similar to that of an immediate-release tablet formulation administered three times daily.

These dosage forms (preferably a tablet or capsule, which may contain beads, granules, particles, or a mixture thereof) may contain baclofen in the amount of from about 2 mg to about 150 mg (preferably from about 2.5 mg to about 100 mg) and can be used in the treatment of medical conditions, which includes spasms, cramping, and tightness of muscles, that are associated with ailments such as multiple sclerosis or certain spinal injuries.

Total daily dosages of the compounds useful according to this invention administered to a host in single or divided doses are generally in amounts of from about 0.01 mg/kg to about 100 mg/kg body weight daily, and preferably from about 0.05 mg/kg to about 50 mg/kg body weight daily. It should be understood, however, that the specific dose level for any particular patient will depend upon a variety of factors including body weight, general health, gender, diet, time and route of administration, rates of absorption and excretion, combination with other drugs, and the severity of the particular disease being treated. Actual dosage levels of active ingredient in the compositions of the present invention may be varied so as to obtain an amount of active ingredient that is effective to obtain a desired therapeutic response for a particular composition and method of administration.

Total daily dose of the compounds useful according to this invention administered to a host in single or divided doses may be in amounts, for example, of from about 0.01 mg/kg to about 20 mg/kg body weight daily and preferably 0.02 to 10 mg/kg/day. The preferred dosage range of baclofen is between 2.5 mg and 100 mg per dosage form. Dosage forms according to the present invention may contain such amounts or fractions thereof as may be used to make up the daily dose.

Preferred dosage strengths for the formulations of the present invention include those having 10 mg, 15 mg, 20 mg, 25 mg, 30 mg, 35 mg and 40 mg baclofen. Typically, the optimal dosage for a patient will be determined by titration, whereby the patient is initially given small doses, which are then gradually increased until the patient reaches the dosage level that achieves maximum therapeutic efficacy with minimum side effects.

One embodiment of the present invention provides a controlled release solid oral dosage form in which there is immediate release of baclofen and delayed or delayed-sustained release of baclofen. Dosages according to the present invention may include an immediate release component and a delayed or delayed-sustained release component. The combination of these two components can release the drug in a pulsed release fashion or a continuous fashion upon oral administration of the dosage form.

In one aspect, the invention relates to a controlled release baclofen solid oral dosage form comprising an immediate release baclofen component and a delayed or delayed-sustained, or sustained release baclofen component. The immediate release baclofen component comprises baclofen formulated with one or more pharmaceutically acceptable excipients that allow for immediate release of the baclofen, and the delayed, or delayed-sustained, or sustained release baclofen component comprises baclofen formulated with one or more excipients that allow for delayed, or delayed-sustained, or sustained release of the baclofen. For example, see U.S. Pat. No. 6,372,254 that refers to formulations, such as tablets, having both an immediate release component and an extended release component.

Among other dosage forms apparent to the skilled artisan, the solid oral dosage form according to the present invention may be a tablet formulation, or a discrete unit-filled capsule formulation, or a sachet formulation. The discrete units of the present invention include beads, granules, pellets, spheroids, particles, tablets, pills, etc.

Specifically, the immediate release, delayed release, delayed-sustained release, and sustained release components of the dosage form can take any form known to a skilled pharmaceutical formulator, including one component of a multi-component tablet such as described in U.S. Pat. 6,372,254, issued Apr. 16, 2002, pending U.S. patent application Ser. No. 10/241,837, filed Sep. 12, 2002, and published International Patent Application No. WO 03/101432, filed Dec. 11, 2003, each assigned to Impax Laboratories, Inc. The controlled release baclofen dosages according to the present invention may be in the form of cores comprising baclofen.

Dosage forms can be made according to known methods in the art. Some preferred methods are described below.

Matrix Dosage Forms. The term matrix, as used herein, refers to a solid material having an active agent incorporated therein. Upon exposure to a dissolution media, channels are formed in the solid material so that the active agent can escape. Dosage forms according to the present invention may be in the form of coated or uncoated matrices. A coating, for example may contain immediate release baclofen, or in the alternative, and the matrix itself can contain controlled release baclofen. Drug release from the delayed or delayed-sustained, or sustained release component can be immediate or sustained, for example within 7 hours after oral administration of the oral dosage form to ensure effective absorption of the drug.

The controlled release baclofen component may be comprised of baclofen coated with at least one delayed release layer. The delayed-sustained release baclofen component may be comprised of sustained-release-coated baclofen coated with at least one delayed release layer. The sustained release baclofen component may be comprised of baclofen coated with at least one sustained-release polymer, or a matrix-controlled release polymer.

The skilled artisan should appreciate that the matrix material can be chosen from a wide variety of materials that can provide the desired dissolution profiles. Materials can include, for example, one or more gel forming polymers such as polyvinyl alcohol, cellulose ethers including, for example, hydroxyl propyl alkyl, celluloses such as hydroxypropyl methyl cellulose, hydroxy alkyl celluloses such as hydroxy propyl cellulose, natural or synthetic gums such as guar gum, xanthum gum, and alginates, as well as, ethyl cellulose, polyethylene oxide, polyvinyl pyrrolidone, fats, waxes, polycarboxylic acids or esters such as the Carbopol® series of polymers, methacrylic acid copolymers, and methacrylate polymers.

Methods of making matrix dosages are known in the art and any such method that can yield the desired dissolution profiles and/or plasma profiles may be relied upon according to the present invention. One such method involves baclofen with a solid polymeric material and one or more pharmaceutically acceptable excipients that are then blended and compressed in controlled release tablet cores. Such tablet cores can be used for further processing as bilayer tablets, press coated tablets, or film coated tablets.

A coating containing the immediate release baclofen can be added to the outside of the controlled release tablet cores to produce a final dosage form. Such a coating can be prepared by mixing baclofen with polyvinylpyrrolidone (PVP) 29/32 or hydroxypropyl methylcellulose (HPMC) and water/isopropyl alcohol and triethyl acetate. Such an immediate release coating can be spray coated onto the tablet cores. The immediate release coating may also be applied using a press-coating process with a blend consisting of 80% by weight baclofen and 20% by weight of lactose and hydroxypropyl methylcellulose type 2910. Press coating techniques are known in the art and are described in U.S. Pat. No. 6,372,254 (Ting et al.), incorporated herein by reference in its entirety.

In addition, the formulation of respective release components can occur by appropriate granulation methods as is well known in the art. In wet granulation, solutions of the binding agent (polymer) are added with stirring to the mixed powders. The powder mass is wetted with the binding solution until the mass has the consistency of damp snow or brown sugar. The wet granulated material is forced through a sieving device. Moist material from the milling step is dried by placing it in a temperature controlled container. After drying, the granulated material is reduced in particle size by passing it through a sieving device. Lubricant is added, and the final blend is then compressed into a matrix dosage form.

In fluid-bed granulation, particles of inert material and/or active agent are suspended in a vertical column with a rising air stream. While the particles are suspended, a common granulating material in solution is sprayed into the column. There is a gradual particle buildup under a controlled set of conditions resulting in tablet granulation. Following drying and the addition of lubricant, the granulated material is ready for compression.

In dry-granulation, the active agent, binder, diluent, and lubricant are blended and compressed into tablets. The compressed large tablets are comminuted through the desirable mesh screen by sieving equipment. Additional lubricant is added to the granulated material and blended gently. The material is then compressed into tablets.

Particle Based Dosage Forms. Immediate Release Particles. The immediate release/controlled release dosage forms of the present invention can also take the form of pharmaceutical particles. The dosage forms can include immediate release particles in combination with controlled release particles in a ratio sufficient to deliver the desired release of active agents. The controlled release particles can be produced by coating the immediate release particles.

The term “particle” as used herein means a granule having a diameter of between about 0.01 mm and about 5.0 mm, preferably between about 0.1 nun and about 2.5 mm, and more preferably between about 0.5 mm and about 2 mm. The skilled artisan should appreciate that particles according to the present invention can be any geometrical shape within this size range and so long as the mean for a statistical distribution of particles falls within the particle sizes enumerated above, they will be considered to fall within the contemplated scope of the present invention. Particles can assume any standard structure known in the pharmaceutical arts. Such structures include, for example, matrix particles, non-pareil cores having a drug layer and active or inactive cores having multiple layers thereon. A controlled release coating can be added to any of these structures to create a controlled release particle.

The particles can be produced according to any of a number of known methods for making particles. The immediate release particles comprise the active agent combination and a disintegrant. Suitable disintegrants include, for example, starch, low-substitution hydroxypropyl cellulose, croscarmellose sodium, calcium carboxymethyl cellulose, hydroxypropyl starch, sodium starch glycolate, and microcrystalline cellulose.

In addition to the above-mentioned ingredients, the matrix may also contain suitable quantities of other materials, for example, diluents, lubricants, binders, granulating aids, colorants, flavorants, and glidants that are conventional in the pharmaceutical arts. The quantities of these additional materials are sufficient to provide the desired effect to the desired formulation. A matrix incorporating particles may also contain suitable quantities of these other materials such as diluents, lubricants, binders, granulating aids, colorants, flavorants, and glidants that are conventional in the pharmaceutical arts in amounts up to about 75% by weight of the particulate, if desired.

In one preferred embodiment, oral dosage forms are prepared to include an effective amount of particles as described above within a capsule. For example, melt-extruded particles may be placed in a gelatin capsule in an amount sufficient to provide an effective controlled release dose when ingested and contacted by gastric fluid. In another preferred embodiment, a suitable amount of the particles are compressed into an oral tablet using conventional tableting equipment using standard techniques. Techniques and compositions for making tablets (compressed and molded), capsules (hard and soft gelatin), and pills are also described in REMINGTON'S PHARMACEUTICAL SCIENCES, Arthur Osol, ed., 1553-93 (1980), incorporated herein by reference. The particles can be made by mixing the relevant ingredients and granulating the mixture. The resulting particles are dried and screened, and the particles having the desired size are used for drug formulation.

Controlled Release Particles. The controlled release particles of the present invention slowly release baclofen when ingested and exposed to gastric fluids, and then to intestinal fluids. The controlled release profile of the formulations of the invention can be altered, for example, by increasing or decreasing the thickness of the retardant coating, i.e., by varying the amount of overcoating. The resultant solid controlled release particles may thereafter be placed in a gelatin capsule in an amount sufficient to provide an effective controlled release dose when ingested and contacted by an environmental fluid, e.g., gastric fluid, intestinal fluid or dissolution media. The particles may be overcoated with an aqueous dispersion of a hydrophobic or hydrophilic material to modify the release profile. The aqueous dispersion of hydrophobic material preferably further includes an effective amount of plasticizer, e.g. triethyl citrate. Preformulated aqueous dispersions of ethylcellulose, such as Aquacoat® or Surelease®, may be used. If Surelease® is used, it is not necessary to separately add a plasticizer.

The release of the therapeutically active agent from the controlled release formulation of the present invention can be further influenced, i.e., adjusted to a desired rate, by the addition of one or more release-modifying agents. The release-modifying agent may be organic or inorganic and include materials that can be dissolved, extracted, or leached from the coating in the environment of use. The pore-formers may comprise one or more hydrophilic materials such as hydroxypropyl methylcellulose. The release-modifying agent may also comprise a semi-permeable polymer. In certain preferred embodiments, the release-modifying agent is selected from hydroxypropyl methylcellulose, lactose, metal stearates, and mixtures thereof.

The controlled-release component may also include a combination of hydrophilic and hydrophobic polymers. In this embodiment, once administered, the hydrophilic polymer dissolves away to weaken the structure of the controlled-release component, and the hydrophobic polymer retards the water penetration and helps to maintain the shape of the drug delivery system.

The hydrophobic material may be selected from the group consisting of alkylcellulose, acrylic and methacrylic acid polymers and copolymers, shellac, zein, hydrogenated castor oil, hydrogenated vegetable oil, or mixtures thereof In certain preferred embodiments, the hydrophobic material is a pharmaceutically acceptable acrylic polymer, including but not limited to acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer, poly(methyl methacrylate), poly(methacrylic acid anhydride), polymethacrylate, polyacrylamide, poly(methacrylic acid anhydride), and glycidyl methacrylate copolymers. In alternate embodiments, the hydrophobic material is selected from materials such as one or more hydroxyalkyl celluloses such as hydroxypropyl methylcellulose. The hydroxyalkyl cellulose is preferably a hydroxy (C₁ to C₆) alkyl cellulose, such as hydroxypropylcellulose, hydroxypropylmethylcellulose, or preferably hydroxyethylcellulose. The amount of the hydroxyalkyl cellulose in the present oral dosage form is determined, inter alia, by the precise rate of active agents desired and may vary from about 1% to about 80%.

In embodiments of the present invention where the coating comprises an aqueous dispersion of a hydrophobic polymer, the inclusion of an effective amount of a plasticizer in the aqueous dispersion of hydrophobic polymer can further improve the physical properties of the film. For example, because ethylcellulose has a relatively high glass transition temperature and does not form flexible films under normal coating conditions, it is necessary to plasticize the ethylcellulose before using it as a coating material. Generally, the amount of plasticizer included in a coating solution is based on the concentration of the film-former, e.g., most often from about 1 percent to about 50 percent by weight of the film-former. Concentration of the plasticizer, however, is preferably determined after careful experimentation with the particular coating solution and method of application.

Examples of suitable plasticizers for ethylcellulose include water-insoluble plasticizers such as dibutyl sebacate, diethyl phthalate, triethyl citrate, tributyl citrate, and triacetin, although other water-insoluble plasticizers (such as acetylated monoglycerides, phthalate esters, castor oil, etc.) may be used. Triethyl citrate is an especially preferred plasticizer for the aqueous dispersions of ethyl cellulose of the present invention.

Examples of suitable plasticizers for the acrylic polymers of the present invention include, but are not limited to, citric acid esters such as triethyl citrate NF XVI, tributyl citrate, dibutyl phthalate, and possibly 1,2-propylene glycol. Other plasticizers which have proved to be suitable for enhancing the elasticity of the films formed from acrylic films such as Eudragit® RL/RS lacquer solutions include polyethylene glycols, propylene glycol, diethyl phthalate, castor oil, and triacetin. Triethyl citrate is an especially preferred plasticizer for aqueous dispersions of ethyl cellulose. It has further been found that addition of a small amount of talc reduces the tendency of the aqueous dispersion to stick during processing and acts a polishing agent.

One commercially available aqueous dispersion of ethylcellulose is Aquacoat® which is prepared by dissolving the ethylcellulose in a water-immiscible organic solvent and then emulsifying the ethylcellulose in water in the presence of a surfactant and a stabilizer. After homogenization to generate submicron droplets, the organic solvent is evaporated under vacuum to form a pseudolatex. The plasticizer is not incorporated into the pseudolatex during the manufacturing phase. Thus, prior to using the pseudolatex as a coating, the Aquacoat® is mixed with a suitable plasticizer.

Another aqueous dispersion of ethylcellulose is commercially available as Surelease® (Colorcon, Inc., West Point, Pa., USA). This product is prepared by incorporating plasticizer into the dispersion during the manufacturing process. A hot melt of a polymer, plasticizer (dibutyl sebacate), and stabilizer (oleic acid) is prepared as a homogeneous mixture which is then diluted with an alkaline solution to obtain an aqueous dispersion which can be applied directly onto substrates.

In one preferred embodiment, the acrylic coating is an acrylic resin lacquer used in the form of an aqueous dispersion, such as that which is commercially available from Rohm Pharma under the trade name Eudragit®. In additional preferred embodiments, the acrylic coating comprises a mixture of two acrylic resin lacquers commercially available from Rohm Pharma under the trade names Eudragit® RL 30 D and Eudragit® RS 30 D. Eudragit® RL 30 D and Eudragit® RS 30 D are copolymers of acrylic and methacrylic esters with a low content of quaternary ammonium groups, the molar ratio of ammonium groups to the remaining neutral (meth)acrylic esters being 1:20 in Eudragit® RL 30 D and 1:40 in Eudragit® RS 30 D. The mean molecular weight is about 150,000 Daltons. The code designations RL (high permeability) and RS (low permeability) refer to the permeability properties of these agents. Eudragit® RL/RS mixtures are insoluble in water and in digestive fluids, however, coatings formed from them are swellable and permeable in aqueous solutions and digestive fluids.

The Eudragit® RL/RS dispersions may be mixed together in any desired ratio in order to ultimately obtain a controlled release formulation having a desirable dissolution profile. Desirable controlled release formulations may be obtained, for instance, from a retardant coating derived from one of a variety of coating combinations, such as 100% Eudragitg RL; 50% Eudragit® RL and 50% Eudragit® RS; or 10% Eudragit® RL and 90% Eudragit® RS. One skilled in the art should recognize that other acrylic polymers may also be used, for example, Eudragit® L. In addition to modifying the dissolution profile by altering the relative amounts of different acrylic resin lacquers, the dissolution profile of the ultimate product may also be modified, for example, by increasing or decreasing the thickness of the retardant coating.

In preferred embodiments of the present invention, the stabilized product is obtained by subjecting the coated substrate to oven curing at a temperature above the T_{g} of the plasticized acrylic polymer for the required time period, the optimum values for temperature and time for the particular formulation being determined experimentally. In certain embodiments of the present invention, the stabilized product is obtained via an oven curing conducted at a temperature of about 45° C. for a time period from about 1 to about 48 hours. It is also contemplated that certain products coated with the controlled release coating of the present invention may require a curing time longer than 24 to 48 hours, e.g., from about 48 to about 60 hours or more.

The coating solutions preferably contain, in addition to the film-former, plasticizer, and solvent system (i.e., water), a colorant to provide elegance and product distinction. Color may be added to the solution of the therapeutically active agent instead of, or in addition to the aqueous dispersion of hydrophobic material. For example, color may be added to Aquacoat® via the use of alcohol or propylene glycol based color dispersions, milled aluminum lakes and opacifiers such as titanium dioxide by adding color with shear to the water soluble polymer solution and then using low shear to the plasticized Aquacoat®. Alternatively, any suitable method of providing color to the formulations of the present invention may be used. Suitable ingredients for providing color to the formulation when an aqueous dispersion of an acrylic polymer is used include titanium dioxide and color pigments, such as iron oxide pigments. The incorporation of pigments, may, however, increase the retardant effect of the coating.

Spheroids or beads coated with the therapeutically active agents can be prepared, for example, by dissolving the therapeutically active agents in water and then spraying the solution onto a substrate, for example, non pareil 18/20 beads, using a Wuster insert. Optionally, additional ingredients are also added prior to coating the beads in order to assist the binding of the active agents to the beads, and/or to color the solution, etc. For example, a product that includes hydroxypropyl methylcellulose with or without colorant (e.g., Opadry®, commercially available from Colorcon, Inc.) may be added to the solution and the solution mixed (e.g., for about 1 hour) prior to application onto the beads. The resultant coated substrate, beads in this example, may then be optionally overcoated with a barrier agent to separate the therapeutically active agent from the hydrophobic controlled release coating. An example of a suitable barrier agent is one that comprises hydroxypropylmethylcellulose. However, any film-former known in the art may be used. It is preferred that the barrier agent does not affect the dissolution rate of the final product.

Press Coated. Pulsatile Dosage Form. In another embodiment of the present invention, baclofen is administered via a press coated, pulsatile drug delivery system suitable for oral administration with a controlled release component, which contains a compressed blend of an active agent and one or more polymers, substantially enveloped by an immediate release component, which contains a compressed blend of the active agent and hydrophilic and hydrophobic polymers. The immediate release component preferably comprises a compressed blend of active agent and one or more polymers with disintegration characteristics such that the polymers disintegrate rapidly upon exposure to the aqueous medium.

The controlled release component preferably comprises a combination of hydrophilic and hydrophobic polymers. In this embodiment, once administered, the hydrophilic polymer dissolves away to weaken the structure of the controlled release component, and the hydrophobic polymer retards the water penetration and helps to maintain the shape of the drug delivery system.

In accordance with the present invention, the term “polymer” includes single or multiple polymeric substances, which can swell, gel degrade or erode on contact with an aqueous environment (e.g., water). Examples include alginic acid, carboxymethylcellulose calcium, carboxymethylcellulose sodium, colloidal silicon dioxide, croscarmellose sodium, crospovidone, guar gum, magnesium aluminum silicate, methylcellulose, microcrystalline cellulose, polacrilin potassium, powdered cellulose, pregelatinized starch, sodium alginate, sodium starch glycolate, starch, ethylcellulose, gelatin, hydroxyethyl cellulose, hydroxypropyl cellulose, hydroxypropyl methylcellulose, polymethacrylates, povidone, pregelatinized starch, shellac, zein, and combinations thereof

The term “hydrophilic polymers” as used herein includes one or more of carboxymethylcellulose, natural gums such as guar gum or gum acacia, gum tragacanth, or gum xanthan, hydroxyethyl cellulose, hydroxypropyl cellulose, hydroxypropyl methylcellulose, methylcellulose, and povidone, of which hydroxypropyl methylcellulose is further preferred. The term “hydrophilic polymers” can also include sodium carboxymethycellulose, hydroxymethyl cellulose, polyethelene oxide, hydroxyethyl methyl cellulose, carboxypolymethylene, polyethelene glycol, alginic acid, gelatin, polyvinyl alcohol, polyvinylpyrrolidones, polyacrylamides, polymethacrylamides, polyphosphazines, polyoxazolidines, poly(hydroxyalkylcarboxylic acids), an alkali metal or alkaline earth metal, carageenate alginates, ammonium alginate, sodium alganate, or mixtures thereof.

The “hydrophobic polymer” of the drug delivery system can be any hydrophobic polymer which will achieve the goals of the present invention including, but not limited to, one or more polymers selected from carbomer, carnauba wax, ethylcellulose, glyceryl palmitostearate, hydrogenated castor oil, hydrogenated vegetable oil type 1, microcrystalline wax, polacrilin potassium, polyethylene oxide, polymethacrylates, or stearic acid, of which hydrogenated vegetable oil type 1 is preferred. Hydrophobic polymers can include, for example, a pharmaceutically acceptable acrylic polymer, including, but not limited to, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methyl methacrylate)copolymer, polyacrylamide, aminoalkyl methacrylate copolymer, poly(methacrylic acid anhydride), and glycidyl methacrylate copolymers. Additionally, the acrylic polymers may be cationic, anionic, or non-ionic polymers and may be acrylates, methacrylates, formed of methacrylic acid or methacrylic acid esters. The polymers may also be pH dependent.

Enteric Coated Controlled Release. In one embodiment, the delayed or delayed-sustained release coating is an enteric coating. All commercially available pH-sensitive polymers may be used to form the enteric coating. The drug coated with the enteric coating is minimally or not released in the acidic stomach environment of approximately below pH 4.5. The drug should become available when the enteric layer dissolves at the higher pH present in the intestine; after a suitable delayed time; or after the unit passes through the stomach. The preferred duration of drug release time is in the range of up to 7 hours after dosing under fasting conditions.

Enteric polymers include cellulose acetate phthalate, cellulose acetate trimellitate, hydroxypropyl methylcellulose phthalate, polyvinyl acetate phthalate, carboxymethylethylcellulose, co-polymerized methacrylic acid/methacrylic acid methyl. esters such as, for instance, materials known under the trade name Eudragit® L12.5, Eudragit® L100, or Eudragit® S12.5, S100 (Rohm GmbH, Darmstadt, Germany) or similar compounds used to obtain enteric coatings. Aqueous colloidal polymer dispersions or re-dispersions can be also applied, e.g., Eudragit® L 30D-55, Eudragit® L100-55, Eudragit® S100, Eudragit® preparation 4110D c; Aquateric®, Aquacoat® CPD 30 (FMC Corp.); Kollicoat MAE® 30D and Kollicoat MAE® 30DP (BASF); Eastacryl® 30D (Eastman Chemical, Kingsport, Tenn.).

The enteric polymers used in this invention can be modified by mixing with other known coating products that are not pH sensitive. Examples of such coating products include the neutral methacrylic acid esters with a small portion of trimethylammonioethyl methacrylate chloride, sold currently under the trade names E Eudragit®, Eudragit® RL, Eudragit® RS; a neutral ester dispersion without any functional groups, sold under the trade names Eudragit® NE30D and Eudragit® NE30; and other pH independent coating products.

The enteric coating will substantially envelop the controlled-release component. The term “substantially envelop” is intended to define the total or near-total enclosure of a component. Such an enclosure includes, preferably, at least about 80% enclosure, more preferably at least about 90% enclosure, and even more preferably at least about 99% enclosure.

In a preferred embodiment, the dosage form is a capsule formulation, which capsule contains a combination of beads containing baclofen in an immediate-release formulation and beads containing baclofen in an enteric-coated controlled-release formulation. In this preferred embodiment, the enteric-coated controlled-release beads contain two layers that control the rate of baclofen release.

The controlled-release beads are prepared by coating the baclofen on sugar spheres, then adding a first polymer layer, followed by a second, pH-sensitive enteric polymer layer. Preferably, the outer, enteric coating will dissolve at pH of about 5.5 or greater. The inner layer may contain a pH-sensitive or pH-independent polymer as described herein, provided that the inner layer functions to provide sustained release of the baclofen upon dissolution of the outer enteric coat. The inner controlled-release polymer layer will be applied in an amount such that, in combination with the outer, enteric-coating, the enteric-coated controlled-release component yields the in vitro dissolution profile described herein. In a particularly preferred embodiment, the inner layer comprises an enteric polymer that dissolves at about pH 6.0. Particularly preferred polymers are described in the Examples that follow.

An embodiment of the present invention provides for a free flowing formulation comprising baclofen. The term “free flowing” as used herein, means dosage forms that pass through a patient's digestive system without impediment or mechanism to slow passage. Thus, for example, the term “free flowing” would exclude gastric raft type dosage forms, which are designed to reside in the stomach for extended periods as in, e.g., U.S. Pat. No. 5,651,985.

Dosage forms according to the present invention can also include a combination of baclofen and at least one additional active agent, such as tizanidine, dantrolene, nonsteroidal anti-inflammatory agents (NSAIDs), opioids, and COX-2 inhibitors. The other active agents can be co-formulated in the immediate-release or delayed-release, delayed-sustained release, or sustained-release components to provide desirable therapeutic effects.

Dosage forms according to the present invention can also apply to pure racemic, L-baclofen, and other GABA related active agents as referred to in U.S. Pat. No. 6,350,769, issued Feb. 26, 2002 to Kaufman et al.

Dosage levels of baclofen (racemic or L-baclofen), as well as any active agent that is to be used in combination with baclofen, in the compositions may be varied so as to obtain an amount of baclofen, and, when used as a combination product, an amount of active ingredient that is effective to obtain a desired therapeutic response for a particular composition and method of administration.

An object of the present invention provides for controlled bioavailability of baclofen as desired by health providers. Bioavailability refers to the degree to which the therapeutically active medicament becomes available in the body after administration. Typically, bioavailability is measured in patients who fasted overnight before being dosed with the test preparation. Plasma samples are then taken and analyzed for the plasma concentration of the parent compound and/or its active metabolite. These data may be expressed as C_{MAX}, the maximum amount of active ingredient found in the plasma, or as AUC, the area under the plasma concentration time curve. Shargel & Yu, APPLIED BIOPHARMACEUTICS AND PHARMACOKINETICS ch. 10 (3d ed. 1996); see also APPLIED PHARMACOKINETICS: PRINCIPLES OF THERAPEUTIC DRUG MONITORING, Evans et al., eds. (3d ed. 1992).

For example, baclofen formulations may be used in a comparative bioavailability study in subjects. Subjects fast over night prior to drug administration. Plasma samples are then taken at dosing, and every hour for twelve hours after dosing, and then at sixteen and twenty-four hours after dosing, and analyzed for the ng/ml concentration of baclofen or a baclofen metabolite.

Without further elaboration, one skilled in the art having the benefit of the preceding description can utilize the present invention to the fullest extent. The following examples are illustrative only and do not limit the remainder of the disclosure in any way.

### EXAMPLES

### Example 1

### Active Baclofen-Coated Seeds

FORMULATION

| INGREDIENT | % | mg |
|---|---|---|
| Sugar Spheres, NF (mesh 20-25) | 81.4 | 250.0 |
| Micronized Baclofen, USP | 13.0 | 40.0 |
| Povidone, USP (Plasdone K-29/32) | 5.6 | 17.14 |
| Purified Water, USP | N/A | N/A |
| TOTAL: | 100.0 | 307.14 |

Povidone (Plasdone K-29/32®) is added to purified water and mixed until the povidone is fully dissolved. Baclofen is mixed in the above solution until uniformly dispersed. A fluidized bed coating apparatus is then used to coat the sugar spheres with the baclofen suspension to produce active coated seeds.

### Example 2

### Active Baclofen-Coated Seeds

FORMULATION

| INGREDIENT | % | mg |
|---|---|---|
| Sugar Spheres, NF (mesh 20-25) | 81.4 | 250.0 |
| Micronized Baclofen, USP | 13.0 | 40.0 |
| Hypromellose, Type 2910, USP | 5.6 | 17.14 |
| (Pharmacoat 606, 6 cps) | | |
| Purified Water, USP | N/A | N/A |
| TOTAL: | 100.0 | 307.14 |

Hypromellose, Type ₂₉₁₀®, USP (Pharmacoat 606, 6cps) is added to a suitable amount of purified water and mixed until the Hypromellose is fully dissolved. Baclofen is mixed in the above solution until uniformly dispersed. A fluidized bed coating apparatus is then used to coat the sugar spheres with the baclofen suspension to produce active coated seeds.

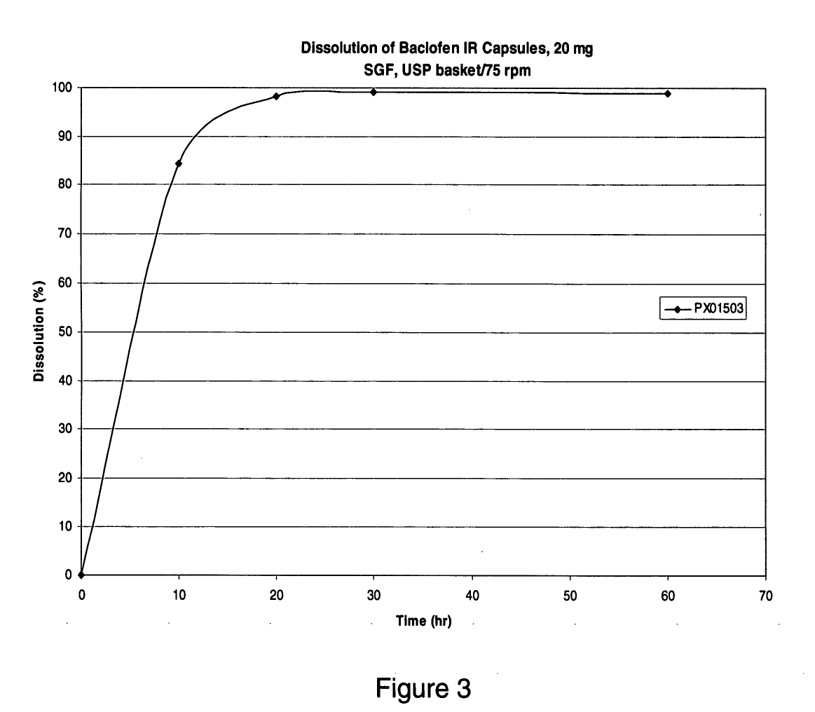

The dissolution profile of this formulation is shown in FIG. 3.

### Example 3

### Active Baclofen-Containing Granules

FORMULATION

| INGREDIENT | % | mg |
|---|---|---|
| Baclofen, USP | 7.4 | 20.0 |
| Pregelatinized Starch, NF | 21.3 | 57.5 |
| (Starch 1500) | | |
| Microcrystalline Cellulose, NF | 70.8 | 191.3 |
| (Avicel PH-102) | | |
| Magnesium Stearate, NF | 0.5 | 1.3 |
| Purified Water, USP | N/A | N/A |
| TOTAL: | 100.0 | 270.1 |

Mix Baclofen, Starch 1500 (pregelatinized starch) and Avicel PH-102 (microcrystalline cellulose). Charge the baclofen mixture into a Hobart mixer and blend to form a uniform mixture. Granulate the mixture with purified water to form a granulate. Dry the granulate in an oven at a temperature of 60° C. to form granules. Screen the granules using a #30 mesh screen. Mix magnesium stearate to form active granules.

### Example 4

### Enteric-Coated Seeds Containing Baclofen

FORMULATION

| INGREDIENT | % | mg |
|---|---|---|
| Active coated seeds | 76.5 | 153.61 |
| (containing 13.02% Baclofen) | | |
| Hypromellose, Type 2910, USP | 8.5 | 17.07 |
| (Pharmacoat 606, 6 cps) | | |
| Hypromellose Phthalate, NF | 13.5 | 27.11 |
| (HPMCP; HP-50) | | |
| Acetyltributyl Citrate, NF | 1.5 | 3.01 |
| Acetone, NF | N/A | N/A |
| Purified Water, USP | N/A | N/A |
| TOTAL: | 100.0 | 200.8 |

Charge purified water into a stainless steel container and mix in hypromellose until completely dissolved. Then charge purified water and acetone into another stainless steel container and then mix in acetyltributyl citrate to form an acetyltributyl citrate solution. To this, add hypromellose phthalate to form an enteric coat solution.

Film coat the baclofen active coated seeds as produced in any of examples 1-3 with the seal coat solution to form sealed baclofen beads. Then film coat the sealed baclofen beads with the enteric coat solution to produce enteric-coated seeds.

### Example 5

### Enteric-Coated Seeds Containing Baclofen

FORMULATION

| | A | | B | |
|---|---|---|---|---|
| INGREDIENT | % | mg | % | mg |
| Active coated seeds | 90.0 | 149.4 | 90.0 | 149.4 |
| (containing 13.42% Baclofen) | | | | |
| Methacrylic Acid Copolymer Type | 8.0 | 13.28 | — | — |
| A, NF (Eudragit L 100) | | | | |
| Methacrylic Acid Copolymer Type | — | — | 8.0 | 13.28 |
| C, NF (Eudragit L 100-55) | | | | |
| Talc, USP | 1.0 | 1.66 | 1.0 | 1.66 |
| Triethyl Citrate, NF | 1.0 | 1.66 | 1.0 | 1.66 |
| Isopropyl Alcohol, USP | N/A | N/A | N/A | N/A |
| Purified Water, USP | N/A | N/A | N/A | N/A |
| TOTAL: | 100.0 | 166.00 | 100.0 | 166.0 |

Charge isopropyl alcohol and purified water into a stainless steel container and then mix in triethyl citrate. Add in methacrylic acid copolymer Type A, NF (Eudragit® L 100) or methacrylic acid copolymer Type C, NF (Eudragit® L 100-55) to form a Eudragit® suspension. Disperse talc into the Eudragit® suspension. Film coat the baclofen active coated seeds from example 4 with the Eudragit® suspension to form enteric-coated seeds.

### Example 6

### Composition Containing Baclofen Active Coated and Enteric-Coated Seeds

FORMULATION

| | Immediate | | Delayed | | | |
|---|---|---|---|---|---|---|
| | release | | release | | | |
| Ingredient | component | | component | | TOTAL | |
| Baclofen | 10 | mg | 20 | mg | 30 | mg |
| Pharmacoat 606 | 2 | mg | 4 | mg | 6 | mg |
| Talc | 0.4 | mg | 12.1 | mg | 12.5 | mg |
| Sugar Spheres | 62.5 | mg | 125 | mg | 187.5 | mg |
| Eudragit L100-55 | 0 | | 22.32 | mg | 22.32 | mg |
| Triethyl Citrate | 0 | | 3.72 | mg | 3.72 | mg |
| Water | N/A | | N/A | | N/A | |
| Isopropyl Alcohol | N/A | | N/A | | N/A | |
| Acetone | N/A | | N/A | | N/A | |
| TOTAL: | 74.9 | | 187.14 | | 262.04 | |

Designated portions of active coated seeds and enteric-coated seeds are mixed together to form dosage forms. In the case of capsules, the seeds are mixed and added to gelatin capsules. In the case of tablets, the seeds are compressed to form a tablet. In the case of sachets, the seed are mixed and filled into the pouch.

### Example 7

### Enteric-Coated Seeds Containing Baclofen

FORMULATION

| INGREDIENT | Weight % |
|---|---|
| Baclofen | 10.56 |
| Sugar Spheres | 65.97 |
| Pharmacoat 606 | 4.52 |
| Eudragit ® RL 100 | 0.60 |
| Eudragit ® RS 100 | 1.39 |
| Dibutyl Sebacate | 0.20 |
| Talc | 1.39 |
| Magnesium Stearate | 0.40 |
| HPMCP HP-50 | 13.50 |
| Triethyl Citrate | 1.50 |
| TOTAL: | 100.00 |

Pharmacoat 606 is dissolved in purified water and baclofen is then dispersed into this aqueous solution to make an aqueous suspension. A fluidized bed coating equipment is used to coat the sugar sphere with the baclofen suspension to produce active coated seeds.

Eudragit® RL 100, RS 100, and dibutyl sebacate are dissolved in a mixture of acetone and isopropyl alcohol. Talc and magnesium stearate are then dispersed into the solution. A fluidized bed coating equipment is used to coat the active coated seeds with the above suspension to produce sustained-release coated seeds.

HPMCP and triethyl citrate are dissolved in a mixture of acetone and purified water. A fluidized bed coating equipment is used to coat the sustained-release coated seeds with the above solution to produce enteric-coated seeds.

### Example 8

### Baclofen Tablets

FORMULATION

| INGREDIENT | Weight (mg) |
|---|---|
| Baclofen | 20 |
| Sodium Starch Glycolate | 20 |
| Dicalcium Phosphate Anhydrous | 26.5 |
| Lactose Anhydrous | 132.5 |
| Mg stearate | 1 |
| TOTAL: | 200 |

Mix baclofen, sodium starch glycolate, dicalcium phosphate anhydrous, and lactose anhydrous in a high-shear granulator. Wet granulate the mixture with purified water and dry the granulate in an oven at a temperature of 60° C. for at least 16 hours. Screen the granules using a #25 mesh screen. Mill the oversized granules by a Fitzpatric comminuting machine equipped with a #18 mesh screen. Blend the screened and milled granules with magnesium stearate and compress the blend into tablets using a rotary tablet press.

### Example 9

### Baclofen Tablets

FORMULATION

| INGREDIENT | Weight (mg) |
|---|---|
| Baclofen | 20 |
| Hydroxypropyl Methylcellulose, type | 60 |
| 2910, USP (Methocel K100LV) | |
| Lactose Monohydrate or Mannitol | 39.60 |
| Microcrystalline Cellulose, NF (Avicel | 79.40 |
| PH101 | |
| Magnesium Stearate | 1.00 |
| TOTAL: | 200 |

Mix baclofen, hydroxypropyl methylcellulose, lactose monohydrate or mannitol, and microcrystalline cellulose in a high-shear granulator. Wet granulate the mixture with purified water and dry the granulate in an oven at a temperature of 60° C. for at least 16 hours. Screen the granules using a #25 mesh screen. Mill the oversized granules by a Fitzpatric comminuting machine equipped with a #18 mesh screen. Blend the screened and milled granules with magnesium stearate and compress the blend into tablets using a rotary tablet press.

### Example 10

### Composition Containing Baclofen Active Coated and Enteric-Coated Seeds

Formulation

| | IR Per | | EC Per | | Total Per | |
|---|---|---|---|---|---|---|
| | Capsule | | Capsule | | Capsule | |
| | % | Amount | % | Amount | % | Amount |
| Ingredient | (w/w) | (mg) | (w/w) | (mg) | (w/w) | (mg) |
| Micronized Baclofen | 13.36 | 19.00 | 21.87 | 21.00 | 16.79 | 40.00 |
| Sugar Spheres, NF (Mesh | 83.48 | 118.73 | 34.11 | 32.75 | 63.58 | 151.48 |
| 20-25) | | | | | | |
| Hypromellose, Type 2910, | 2.67 | 3.80 | 4.37 | 4.20 | 3.36 | 8.00 |
| USP (Pharmacoat 606, 6 cps) | | | | | | |
| Talc, USP (ALTALC | 0.49 | 0.70 | 9.60 | 9.22 | 4.16 | 9.92 |
| 500V USP BC (*1814)) | | | | | | |
| Methacrylic Acid | — | — | 15.53 | 14.91 | 6.26 | 14.91 |
| Copolymer, Type C, NF | | | | | | |
| (Eudragit L100-55) | | | | | | |
| Methacrylic Acid | — | — | 10.61 | 10.19 | 4.28 | 10.19 |
| Copolymer, Type A, NF | | | | | | |
| (Eudragit L100-55) | | | | | | |
| Triethyl Citrate NF | — | — | 3.91 | 3.75 | 1.57 | 3.75 |
| Total | 100.0 | 142.23 | 100.00 | 96.02 | 100.00 | 238.25 |

Hypromellose, Type 2910, USP is added to a suitable amount of purified water and mixed until the hypromellose is fully dissolved. Baclofen is then mixed in the above solution until uniformly dispersed. The suspension is passed through a #40 mesh sieve into a stainless steel container. Sugar spheres are charged into a fluid-bed coater equipped with a Wurster insert and heated until exhaust air temperature reaches 50±5° C. The active suspension from above is sprayed to coat the sugar spheres, which are then dried at a temperature of 60±10° C. for 5-30 minutes. The IR seeds are passed through a #16 mesh stainless steel screen. Acceptable IR seeds are collected and mixed with talc, USP in a slant cone blender for one to ten minutes.

An enteric solution is prepared by mixing isopropyl alcohol and acetone. Triethyl citrate and methacrylic acid copolymer, type A, are stirred into the mixture until completely dissolved. Talc is mixed in the above solution until completely dispersed. A fluidized bed coating apparatus is then used to coat IR seeds prepared as above with the enteric solution to produce enteric-coated seeds. The enteric-coated seeds are passed through a #14 mesh stainless steel screen. Acceptable enteric-coated seeds are collected for second layer enteric-coating.

A second enteric solution is prepared by mixing purified water and acetone. Triethyl citrate and methacrylic acid copolymer, type C, are stirred into the mixture until completely dissolved. Talc is mixed in the above solution until completely dispersed. A fluidized bed coating apparatus is then used to coat enteric-coated seeds prepared as above with the enteric solution to produce the enteric-coated seeds. The enteric-coated seeds are passed through a #12 mesh stainless steel screen. Acceptable enteric-coated seeds are collected and mixed with talc, USP in a slant cone blender for one to ten minutes.

An appropriate amount of IR seeds plus the appropriate amount of enteric-coated seeds are encapsulated to yield Baclofen ER capsules.

### Example 11

### Baclofen Tablets

Tablets having the following compositions were prepared according to the process described in Example 9.

| Ingredients | 579-7C | 579-10A | PX01802 | PX02002 |
|---|---|---|---|---|
| Baclofen, USP | 20 | 20 | 20 | 20 |
| Hydroxypropyl | 0 | 60 | 0 | 0 |
| Methylcellulose, type | | | | |
| 2910, USP(Pharmacoat | | | | |
| 606) | | | | |
| Hydroxypropyl | 20 | 0 | 0 | 0 |
| Methylcellulose, type | | | | |
| 2280, USP(Methocel | | | | |
| K100LV) | | | | |
| Hydroxypropyl | 0 | 0 | 60 | 120 |
| Methylcellulose, type | | | | |
| 2280, USP(Methocel | | | | |
| K100M) | | | | |
| Microcrystalline | 59 | 59 | 59 | 59 |
| Cellulose, NF (Avicel | | | | |
| PH101) | | | | |
| Mg stearate, NF | 1 | 1 | 1 | 1 |
| Purified Water, USP | — | — | — | — |
| tablet wt. | 100 | 140 | 140 | 200 |

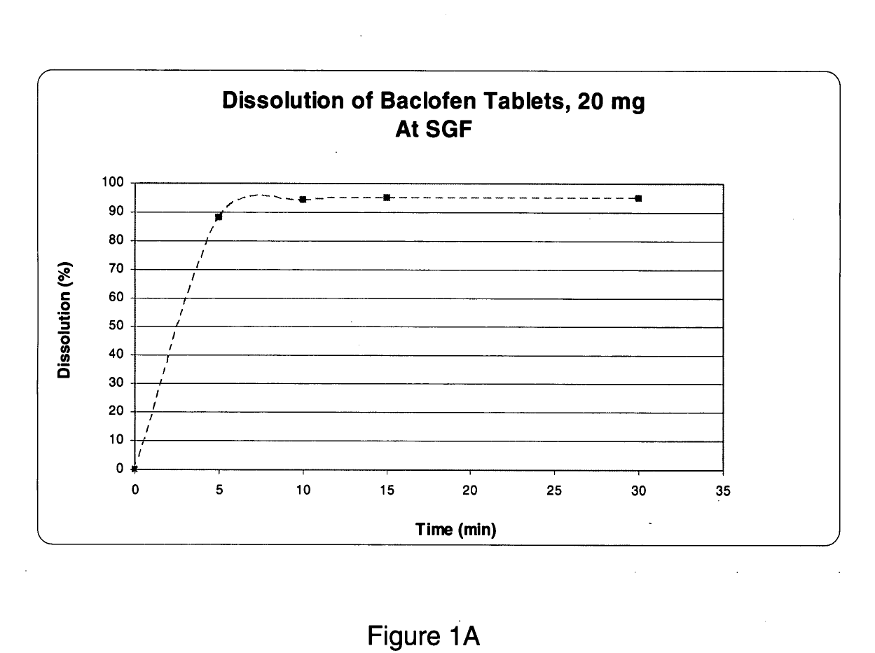

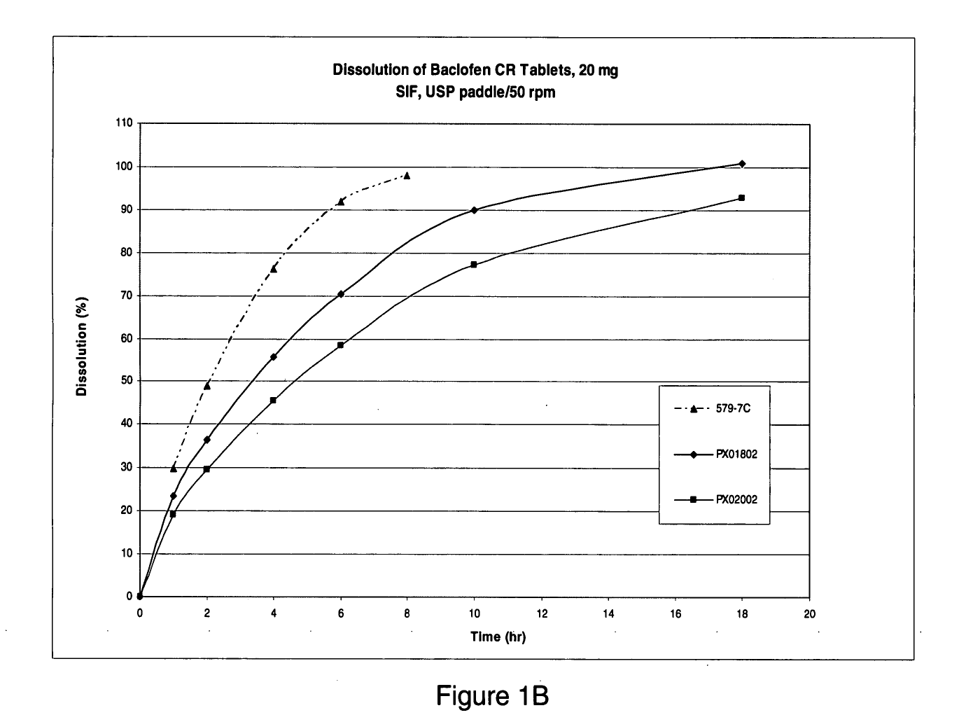

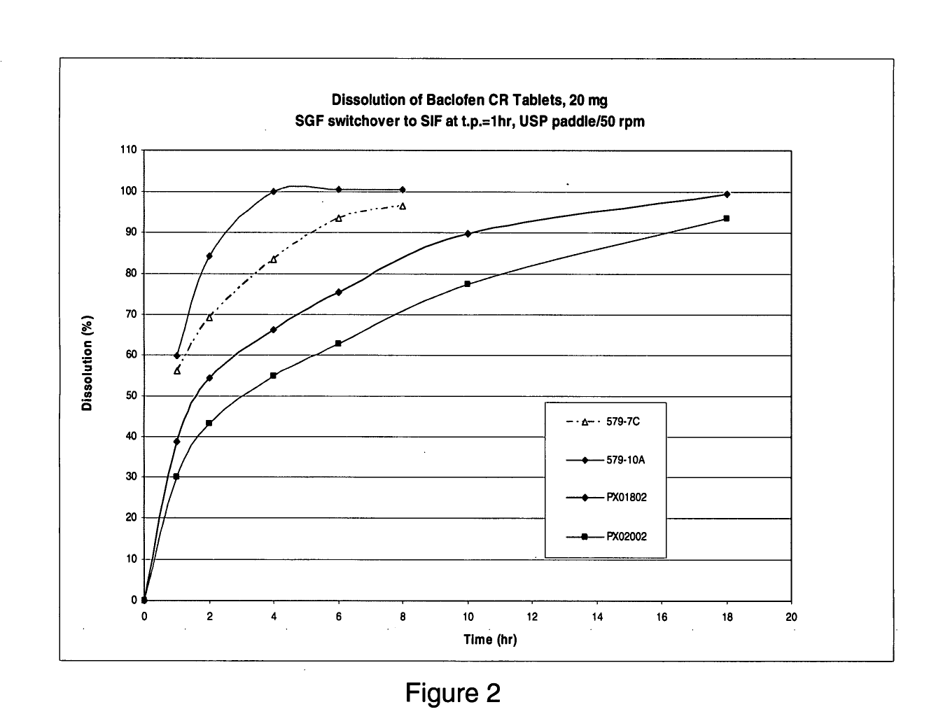

Dissolution profiles of the above formulations are shown in FIG. 1B (in SIF) and in FIG. 2 (SGF/SIF switchover method).

### Example 12

### Baclofen ER Capsules

Baclofen extended release capsules (20 mg) were prepared having the following formulations, using the process described in Example 7.

| Ingredients | PX01903 | PX02103 | PX02503 | PB01403 | PB00903(A) |
|---|---|---|---|---|---|
| Micronized Baclofen, | 20.0 | 20.0 | 20.0 | 20 | 20 |
| USP | | | | | |
| Sugar Spheres | 125.0 | 125.0 | 125.0 | 125.0 | 125.0 |
| Hydroxypropyl | 8.6 | 8.6 | 8.6 | 4.0 | 4.0 |
| Methylcellulose, type | | | | | |
| 2910, USP(Pharmacoat | | | | | |
| 606) | | | | | |
| Hypromellose | 24.4 | 25.6 | 25.6 | 0.0 | 0.0 |
| Phthalate, NF | | | | | |
| (HPMCP; HP-50) | | | | | |
| Ammonio | 0.0 | 1.5 | 1.1 | 0.0 | 0.0 |
| Methacrylate | | | | | |
| Copolymer Type A, NF | | | | | |
| (Eudragit RL100) | | | | | |
| Ammonio | 0.0 | 2.3 | 2.6 | 0.0 | 0.0 |
| Methacrylate | | | | | |
| Copolymer Type B, NF | | | | | |
| (Eudragit RS100) | | | | | |
| Methacrylic Acid | 0.0 | 0.0 | 0.0 | 13.2 | 13.2 |
| Copolymer, Type A, | | | | | |
| NF (Eudragit L100) | | | | | |
| Methacrylic Acid | 0.0 | 0.0 | 0.0 | 8.1 | 0.0 |
| Copolymer, Type C, | | | | | |
| NF (Eudragit L100-55) | | | | | |
| Triethyl Citrate, NF | 2.7 | 2.8 | 2.8 | 3.0 | 3.0 |
| Dibutyl Sebacate, NF | 0.0 | 0.4 | 0.4 | 0.0 | 0.0 |
| Talc, USP | 0.0 | 2.6 | 2.6 | 5.7 | 5.7 |
| Magnesium Stearate, | 0.0 | 0.8 | 0.8 | 0.0 | 0.0 |
| NF | | | | | |
| Isopropyl Alcohol, | 0.0 | — | — | — | — |
| USP | | | | | |
| Acetone, NF | — | — | — | — | — |
| Purified Water, USP | — | — | — | — | — |

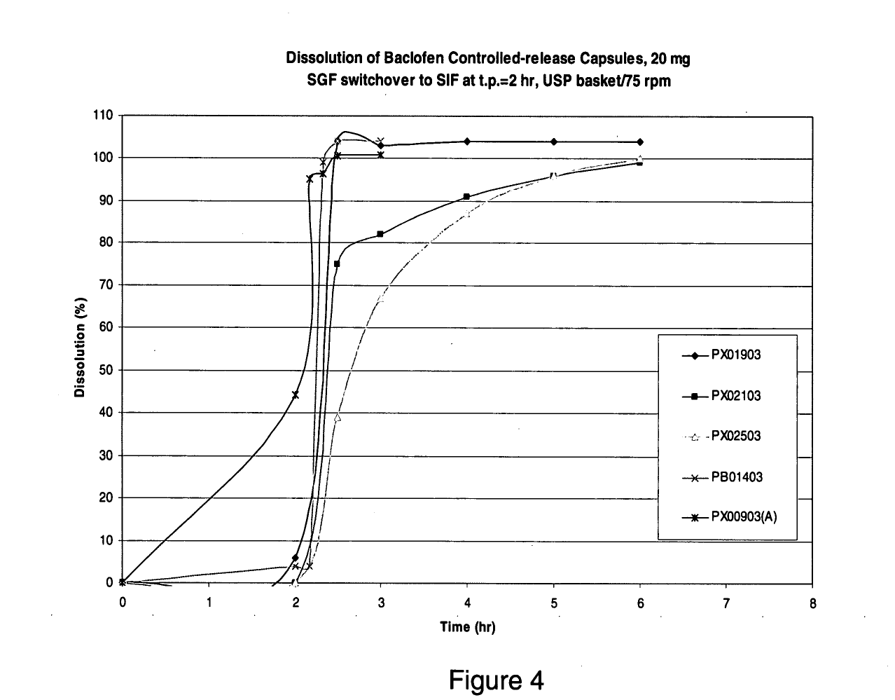

Dissolution profiles of the above formulations are shown in FIG. 4.

### Example 13

### Baclofen ER Capsules

Baclofen ER capsules having the following formulations were prepared according to the process described in Example 10.

Composition of Baclofen ER (ER1A) Capsules
30 mg (Lot PB01903) IR/ER (EC1) = 2:1

| | IR Per | | EC1 Per | | Total Per | |
|---|---|---|---|---|---|---|
| | Capsule | | Capsule | | Capsule | |
| | % | Amount | % | Amount | % | Amount |
| Ingredient | (w/w) | (mg) | (w/w) | (mg) | (w/w) | (mg) |
| Micronized Baclofen | 8.22 | 20.0 | 4.11 | 10.0 | 12.33 | 30.0 |
| Sugar Spheres, NF (Mesh 20-25) | 51.36 | 125.0 | 25.68 | 62.5 | 77.04 | 187.5 |
| Hypromellose, Type 2910, USP | 1.64 | 4.0 | 0.82 | 2.0 | 2.47 | 6.0 |
| (Pharmacoat 606, 6 cps) | | | | | | |
| Talc, USP (ALTALC 500V USP BC | 0.33 | 0.8 | 2.49 | 6.05 | 2.81 | 6.85 |
| (*1814)) | | | | | | |
| Methacrylic Acid Copolymer, Type C, | — | — | 4.59 | 11.16 | 4.59 | 11.16 |
| NF (Eudragit L100-55) | | | | | | |
| Triethyl Citrate NF | — | — | 0.76 | 1.86 | 0.76 | 1.86 |
| Total | 61.55 | 149.8 | 38.45 | 93.57 | 100.00 | 243.37 |

Composition of Baclofen ER (ER1B) Capsules
30 mg (Lot PB01803) IR/ER (EC1) = 1:2

| | IR Per | | EC1 Per | | Total Per | |
|---|---|---|---|---|---|---|
| | Capsule | | Capsule | | Capsule | |
| | % | Amount | % | Amount | % | Amount |
| Ingredient | (w/w) | (mg) | (w/w) | (mg) | (w/w) | (mg) |
| Micronized Baclofen | 3.82 | 10.0 | 7.63 | 20.0 | 11.45 | 30.0 |
| Sugar Spheres, NF (Mesh 20-25) | 23.85 | 62.5 | 47.70 | 125.0 | 71.55 | 187.5 |
| Hypromellose, Type 2910, USP | 0.76 | 2.0 | 1.53 | 4.0 | 2.29 | 6.0 |
| (Pharmacoat 606, 6 cps) | | | | | | |
| Talc, USP (ALTALC 500V USP BC | 0.15 | 0.4 | 4.62 | 12.1 | 4.77 | 12.5 |
| (*1814)) | | | | | | |
| Methacrylic Acid Copolymer, Type | — | — | 8.52 | 22.32 | 8.52 | 22.32 |
| C, NF (Eudragit L100-55) | | | | | | |
| Triethyl Citrate NF | — | — | 1.42 | 3.72 | 1.42 | 3.72 |
| Total | 28.58 | 74.9 | 71.42 | 187.14 | 100.00 | 262.04 |

Composition of Baclofen ER (ER2A) Capsules
30 mg (Lot PB02003) IR/ER (EC2) = 2:1

| | IR Per | | EC2 Per | | Total Per | |
|---|---|---|---|---|---|---|
| | Capsule | | Capsule | | Capsule | |
| | % | Amount | % | Amount | % | Amount |
| Ingredient | (w/w) | (mg) | (w/w) | (mg) | (w/w) | (mg) |
| Micronized Baclofen | 8.34 | 20.0 | 4.17 | 10.0 | 12.51 | 30.0 |
| Sugar Spheres, NF (Mesh 20-25) | 52.14 | 125.0 | 26.07 | 62.5 | 78.21 | 187.5 |
| Hypromellose, Type 2910, USP | 1.67 | 4.0 | 0.83 | 2.0 | 2.50 | 6.0 |
| (Pharmacoat 606, 6 cps) | | | | | | |
| Talc, USP (ALTALC 500V USP BC | 0.33 | 0.8 | 1.38 | 3.29 | 1.71 | 4.09 |
| (*1814)) | | | | | | |
| Methacrylic Acid Copolymer, Type | — | — | 1.68 | 4.03 | 1.68 | 4.03 |
| C, NF (Eudragit L100-55) | | | | | | |
| Methacrylic Acid Capalymer, Type | — | — | 2.76 | 6.62 | 2.76 | 6.62 |
| A, NF (Eudragit L100) | | | | | | |
| Triethyl Citrate NF | — | — | 0.63 | 1.5 | 0.63 | 1.5 |
| Total | 62.48 | 149.8 | 37.52 | 89.94 | 100.00 | 239.74 |

Composition of Baclofen ER (ER2B) Capsules
30 mg (Lot PB02103) IR/ER (EC2) = 1:2

| | IR Per | | EC2 Per | | Total Per | |
|---|---|---|---|---|---|---|
| | Capsule | | Capsule | | Capsule | |
| | % | Amount | % | Amount | % | Amount |
| Ingredient | (w/w) | (mg) | (w/w) | (mg) | (w/w) | (mg) |
| Micronized Baclofen | 3.92 | 10 | 7.85 | 20.0 | 11.78 | 30.0 |
| Sugar Spheres, NF (Mesh 20-25) | 24.53 | 62.5 | 49.06 | 125.0 | 73.59 | 187.5 |
| Hypromellose, Type 2910, USP | 0.78 | 2.0 | 1.57 | 4.0 | 2.35 | 6.0 |
| (Pharmacoat 606, 6 cps) | | | | | | |
| Talc, USP (ALTALC 500V USP BC | 0.16 | 0.4 | 2.58 | 6.58 | 2.74 | 6.98 |
| (*1814)) | | | | | | |
| Methacrylic Acid Copolymer, Type | — | — | 3.16 | 8.06 | 3.16 | 8.06 |
| C, NF (Eudragit L100-55) | | | | | | |
| Methacrylic Acid Copolymer, Type | — | — | 5.20 | 13.24 | 5.20 | 13.24 |
| A, NF (Eudragit L100) | | | | | | |
| Triethyl Citrate NF | — | — | 1.18 | 3.0 | 1.18 | 3.0 |
| Total | 29.39 | 74.9 | 70.60 | 179.88 | 100.00 | 254.78 |

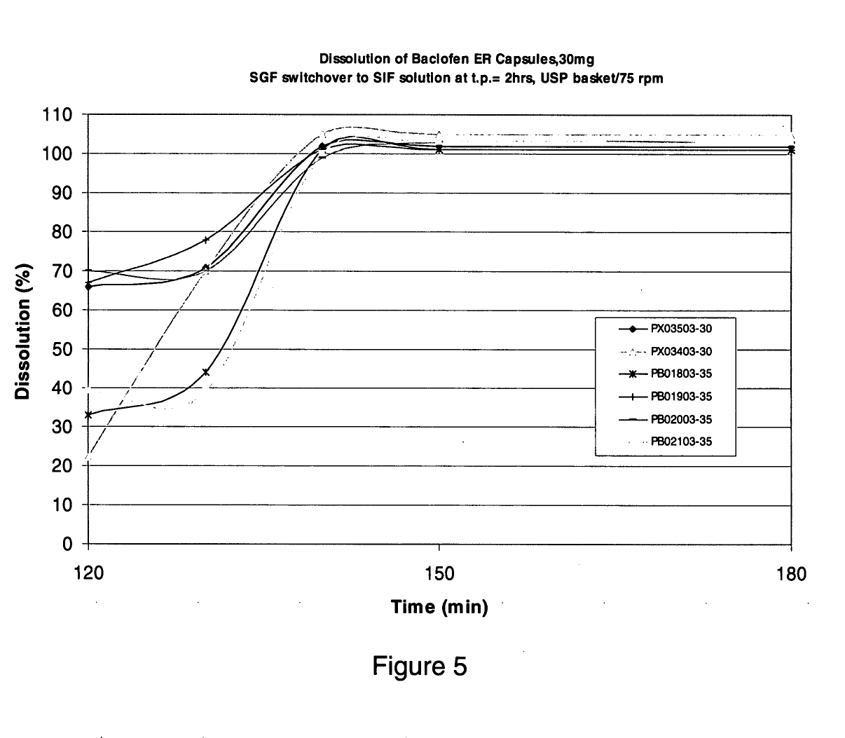

The dissolution profile of the above formulations is shown in FIG. 5.

### Example 14

### Baclofen ER Capsules

Baclofen ER capsules of the following compositions were prepared according to the process described in Example 10, with the exception that the enteric materials are Acetyltributyl Citrate and Hypromellose Phthalate instead of Triethyl Citrate and Methacrylic Acid Copolymer, type C.

PX03503-30 Formulation

| | IR Per | | EC Per | | Total Per | |
|---|---|---|---|---|---|---|
| | Capsule | | Capsule | | Capsule | |
| | % | Amount | % | Amount | % | Amount |
| Ingredient | (w/w) | (mg) | (w/w) | (mg) | (w/w) | (mg) |
| Micronized | 12.37 | 20.00 | 9.17 | 10.00 | 11.06 | 30.0 |
| Balcofen | | | | | | |
| Sugar Spheres, | 77.33 | 125.03 | 57.31 | 62.50 | 69.12 | 187.53 |
| NF (Mesh | | | | | | |
| 20-25) | | | | | | |
| Hypromellose, | 10.30 | 16.65 | 7.63 | 8.32 | 9.20 | 24.97 |
| Type 2910, | | | | | | |
| USP | | | | | | |
| (Pharmacoat | | | | | | |
| 606, 6 cps) | | | | | | |
| Hypromellose | — | — | 11.77 | 12.83 | 4.73 | 12.83 |
| Phthalate, NF | | | | | | |
| (HPMCP; | | | | | | |
| HP-55) | | | | | | |
| Acetyltributyl | — | — | 0.47 | 0.51 | 0.19 | 0.51 |
| Citrate, NF | | | | | | |
| Talc, USP | — | — | 13.65 | 14.89 | 5.70 | 15.46 |
| Total | 100.0 | 161.68 | 100.0 | 109.05 | 100.0 | 271.30 |

PX03403-30 Formulation

| | IR Per | | EC Per | | Total Per | |
|---|---|---|---|---|---|---|
| | Capsule | | Capsule | | Capsule | |
| | % | Amount | % | Amount | % | Amount |
| Ingredient | (w/w) | (mg) | (w/w) | (mg) | (w/w) | (mg) |
| Micronized | 12.37 | 6.00 | 9.17 | 24.00 | 9.65 | 30.0 |
| Balcofen | | | | | | |
| Sugar Spheres, | 77.32 | 37.50 | 57.31 | 149.98 | 60.30 | 187.48 |
| NF (Mesh | | | | | | |
| 20-25) | | | | | | |
| Hypromellose, | 10.31 | 5.00 | 7.63 | 19.98 | 8.03 | 24.98 |
| Type 2910, | | | | | | |
| USP | | | | | | |
| (Pharmacoat | | | | | | |
| 606, 6 cps) | | | | | | |
| Hypromellose | — | — | 11.77 | 30.80 | 9.91 | 30.80 |
| Phthalate, NF | | | | | | |
| (HPMCP; | | | | | | |
| HP-55) | | | | | | |
| Acetyltributyl | — | — | 0.47 | 1.23 | 0.40 | 1.23 |
| Citrate, NF | | | | | | |
| Talc, USP | — | — | 13.65 | 35.73 | 11.71 | 36.41 |
| Total | 100.0 | 48.50 | 100.0 | 261.72 | 100.0 | 310.9 |

Dissolution profiles of the above formulations are shown in FIG. 5.

### Example 15

### Baclofen ER Capsules

Baclofen ER capsules having the following composition were prepared according to the method described in Example 10. Capsules were prepared having 10 mg, 15 mg, 20 mg, 25 mg, 30 mg, 35 mg and 40 mg baclofen, with the different dosage strengths being directly proportional.

Composition of Baclofen ER Capsules
40 mg (Lot RB04042-60A) IR/EC = 19:21

| | IR Per | | EC Per | | Total Per | |
|---|---|---|---|---|---|---|
| | Capsule | | Capsule | | Capsule | |
| | % | Amount | % | Amount | % | Amount |
| Ingredient | (w/w) | (mg) | (w/w) | (mg) | (w/w) | (mg) |
| Micronized Baclofen | 13.36 | 19.00 | 21.87 | 21.00 | 16.79 | 40.00 |
| Sugar Spheres, NF (Mesh 20-25) | 83.48 | 118.73 | 34.11 | 32.75 | 63.58 | 151.48 |
| Hypromellose, Type 2910, USP | 2.67 | 3.80 | 4.37 | 4.20 | 3.36 | 8.00 |
| (Pharmacoat 606, 6 cps) | | | | | | |
| Talc, USP (ALTALC 500V USP BC | 0.49 | 0.70 | 9.60 | 9.22 | 4.16 | 9.92 |
| (*1814)) | | | | | | |
| Methacrylic Acid Copolymer, Type | — | — | 15.53 | 14.91 | 6.26 | 14.91 |
| C, NF (Eudragit L100-55) | | | | | | |
| Methacrylic Acid Copolymer, Type | — | — | 10.61 | 10.19 | 4.28 | 10.19 |
| A, NF (Eudragit L100-55) | | | | | | |
| Triethyl Citrate NF | — | — | 3.91 | 3.75 | 1.57 | 3.75 |
| Total | 100.0 | 142.23 | 100.00 | 96.02 | 100.00 | 238.25 |

### Example 16

### Determining Plasma Profiles for Baclofen-Containing Formulations

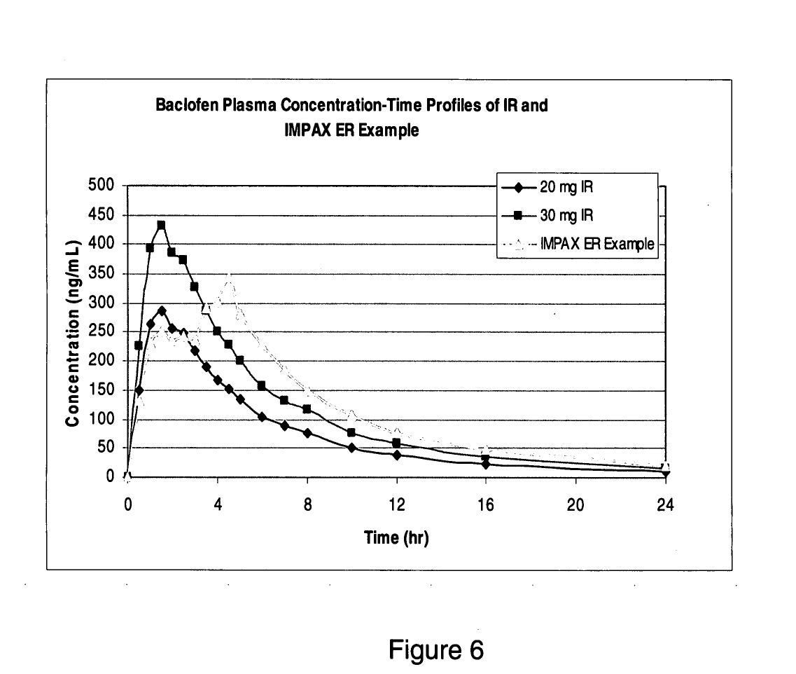

A bioavailability study was done in 20 healthy volunteers comparing a 36 mg baclofen formulation prepared according to Example 15, with the exception that the immediate-release component contained 12 mg baclofen and the enteric-coated controlled release component contained 24 mg baclofen, and the remaining excipients were adjusted dose proportionally. The formulation was compared with a 20 mg immediate release reference tablet (Watson Laboratories, Inc.) under fasting conditions. Test samples were administered orally with 240 ml of room temperature water after subjects are fasted overnight for at least 10 hours. No fluid, except that given with drug administration, is allowed from 1 hour prior to dose administration until 1 hour after dosing. At 2, 6, 8 and 12 hours post-dose, subjects consumed 240 ml of room temperature water. In addition, subjects consumed 480 ml of fluid with lunch and dinner. Blood samples were drawn at 0.5, 1, 1.5, 2, 2.5, 3, 3.5, 4, 4.5, 5, 6, 7, 8, 10, 12, 16, and 24 hours after administration. The results are shown in FIG. 6. In addition, FIG. 6 shows simulated blood plasma levels for 30 mg immediate-release baclofen, based on the data obtained from administration of the 20 mg dosage strength.

### Example 17

### Determining Steady State Plasma Profiles for Baclofen-Containing Formulations

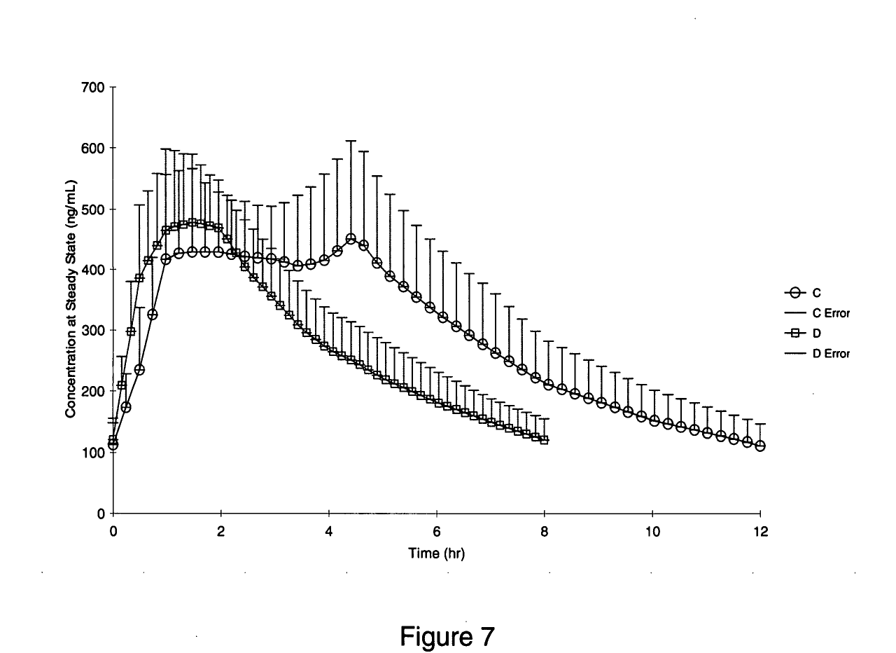

Based on single-dose bioavailability data, steady-state mean baclofen plasma levels were calculated for a 40 mg baclofen formulation prepared according to Example 15 administered every 12 hours and an immediate-release 20 mg baclofen formulation (Watson Laboratories, Inc.) administered every 8 hours. The results are shown in FIG. 7 (where (C) represents the 40 mg dosage form of the present invention and (D) represents the reference 20 mg immediate-release dosage form). The results show that, at steady-state, the 40 mg dosage form of the present invention will reach a C_{MIN} at 12 hours after administration comparable to the C_{MIN} obtained by the immediate-release formulation eight hours after administration.

Having now fully described this invention, it will be understood to those of ordinary skill in the art that the methods of the present invention can be carried out with a wide and equivalent range of conditions, formulations, and other parameters without departing from the scope of the invention or any embodiments thereof.

1. A pharmaceutical dosage form comprising a controlled release component,
wherein said controlled release component comprises a GABA_{B} agonist and a pharmaceutically acceptable excipient; and
wherein said controlled release component exhibits an in vitro dissolution profile in simulated intestinal fluid medium comprising less than about 70% GABA_{B} agonist release after 1 hour, at least about 20% GABA_{B} agonist release after 4 hours, and at least about 30% GABA_{B} agonist release after 6 hours.

2. A pharmaceutical dosage form according to claim 1 wherein the controlled release component exhibits an in vitro dissolution profile in simulated gastric fluid/simulated intestinal fluid (1 hour switchover) medium comprising less than about 80% GABA_{B} agonist release after 1 hour, at least about 30% GABA_{B} agonist release after 4 hours, and at least about 40% GABA_{B} agonist release after 6 hours.

3. A pharmaceutical dosage form according to claim 1 wherein the controlled release component exhibits an in vitro dissolution profile in simulated intestinal fluid medium comprising less than about 50% GABA_{B} agonist release after 1 hour, at least about 40% GABA_{B} agonist release after 4 hours, and at least about 50% GABA_{B} agonist release after 6 hours.

4. A pharmaceutical dosage form according to claim 3 wherein the controlled release component exhibits an in vitro dissolution profile in simulated gastric fluid/simulated intestinal fluid (1 hour switchover) medium comprising less than about 70% GABA_{B} agonist release after 1 hour, at least about 40% GABA_{B} agonist release after 4 hours, and at least about 50% GABA_{B} agonist release after 6 hours.

5. A pharmaceutical dosage form according to claim 1, further comprising an immediate release component comprising a GABA_{B} agonist and a pharmaceutically acceptable excipient;
wherein said immediate release component exhibits an in vitro dissolution profile comprising at least about 80% GABA_{B} agonist release after 1 hour in simulated gastric fluid; and
wherein the ratio of said immediate release component to said controlled release component is from about 1:10 to about 10:1.

6. A pharmaceutical dosage form according to claim 5 wherein said GABA_{B} agonist is baclofen.

7. A pharmaceutical dosage form according to claim 6 wherein the ratio of immediate release component to said controlled release component is from about 1:4 to about 4.

8. A pharmaceutical dosage form according to claim 7 wherein the ratio of immediate release component to said controlled release component is from about 1:2 to about 2:1.

9. A pharmaceutical dosage form according to claim 6 wherein said baclofen is in the amount from about 2 mg to about 150 mg.

10. A pharmaceutical dosage form according to claim 9 wherein said baclofen is in the amount of about 20 mg.

11. A pharmaceutical dosage form according to claim 9 wherein said baclofen is in the amount of about 25 mg.

12. A pharmaceutical dosage form according to claim 9 wherein said baclofen is in the amount of about 30 mg.

13. A pharmaceutical dosage form according to claim 9 wherein said baclofen is in the amount of about 35 mg.

14. A pharmaceutical dosage form according to claim 9 wherein said baclofen is in the amount of about 40 mg.

15. A pharmaceutical dosage form according to claim 6 wherein the baclofen is formulated as a combination of immediate-release beads and controlled-release beads.

16. A pharmaceutical dosage form according to claim 15 wherein said dosage form is a tablet.

17. A pharmaceutical dosage form according to claim 15 wherein said dosage form is a capsule.

18. A pharmaceutical dosage form comprising an enteric-coated controlled release component,
wherein said enteric-coated controlled release component comprises a GABA_{B} agonist and a pharmaceutically acceptable excipient; and
wherein said enteric-coated controlled release component exhibits an in vitro dissolution profile in simulated gastric fluid/simulated intestinal fluid (2 hour switchover) medium comprising less than about 10% GABA_{B} agonist release after 2 hours, at least about 40% GABA_{B} agonist release after 3 hours, and at least about 70% GABA_{B} agonist release after 6 hours.

19. A pharmaceutical dosage form according to claim 18 wherein said enteric-coated controlled release component exhibits an in vitro dissolution profile in simulated gastric fluid/simulated intestinal fluid (2 hour switchover) medium comprising less than about 10% GABA_{B} agonist release after 2 hours, at least about 50% GABA_{B} agonist release after 3 hours, and at least about 80% GABA_{B} agonist release after 6 hours.

20. A pharmaceutical dosage form according to claim 19 wherein said enteric-coated controlled release component exhibits an in vitro dissolution profile in simulated gastric fluid/simulated intestinal fluid (2 hour switchover) medium comprising less than about 10% GABA_{B} agonist release after 2 hours, at least about 60% GABA_{B} agonist release after 3 hours, and at least about 90% GABA_{B} agonist release after 6 hours.

21. A pharmaceutical dosage form according to claim 18, further comprising an immediate release component comprising a GABA_{B} agonist and a pharmaceutically acceptable excipient;
wherein said immediate release component exhibits an in vitro dissolution profile comprising at least about 80% GABA_{B} agonist release after 1 hour in simulated gastric fluid; and
wherein the ratio of said immediate release component to said controlled release component is from about 1:10 to about 10:1.

22. A pharmaceutical dosage form according to claim 21 wherein said GABA_{B} agonist is baclofen.

23. A pharmaceutical dosage form according to claim 22 wherein the ratio of immediate release component to said controlled release component is from about 1:4 to about 4:1.

24. A pharmaceutical dosage form according to claim 23 wherein the ratio of immediate release component to said controlled release component is from about 1:2 to about 2:1.

25. A pharmaceutical dosage form according to claim 22 wherein said baclofen is in the amount from about 2 mg to about 150 mg.

26. A pharmaceutical dosage form according to claim 25 wherein said baclofen is in the amount of about 20 mg.

27. A pharmaceutical dosage form according to claim 25 wherein said baclofen is in the amount of about 25 mg.

28. A pharmaceutical dosage form according to claim 25 wherein said baclofen is in the amount of about 30 mg.

29. A pharmaceutical dosage form according to claim 25 wherein said baclofen is in the amount of about 35 mg.

30. A pharmaceutical dosage form according to claim 25 wherein said baclofen is in the amount of about 40 mg.

31. A pharmaceutical dosage form according to claim 21 wherein the baclofen is formulated as a combination of immediate-release beads and controlled-release beads.

32. A pharmaceutical dosage form according to claim 31 wherein said dosage form is a tablet.

33. A pharmaceutical dosage form according to claim 31 wherein said dosage form is a capsule.

34. A pharmaceutical dosage form comprising a GABA_{B} agonist and a pharmaceutically acceptable excipient, wherein said pharmaceutical dosage form exhibits an in vitro dissolution profile in simulated gastric fluid/simulated intestinal fluid (2 hour switchover) medium comprising less than about 75% GABA_{B} agonist release after 2 hours, and at least about 80% GABA_{B} agonist release after 3 hours.

35. A pharmaceutical dosage form according to claim 34 wherein the pharmaceutical dosage form exhibits an in vitro dissolution profile in simulated gastric fluid/simulated intestinal fluid (2 hour switchover) medium comprising less than about 65% GABA_{B} agonist release after 2 hours, and at least about 90% GABA_{B} agonist release after 3 hours.

36. A pharmaceutical dosage form according to claim 34 wherein the GABA_{B} agonist is baclofen.

37. A pharmaceutical dosage form according to claim 36 wherein said baclofen is in the amount from about 2 mg to about 150 mg.

38. A pharmaceutical dosage form according to claim 37 wherein said baclofen is in the amount of about 20 mg.

39. A pharmaceutical dosage form according to claim 37 wherein said baclofen is in the amount of about 25 mg.

40. A pharmaceutical dosage form according to claim 37 wherein said baclofen is in the amount of about 30 mg.

41. A pharmaceutical dosage form according to claim 37 wherein said baclofen is in the amount of about 35 mg.

42. A pharmaceutical dosage form according to claim 37 wherein said baclofen is in the amount of about 40 mg.

43. A pharmaceutical dosage form according to claim 36 wherein the baclofen is formulated as a combination of immediate-release beads and controlled-release beads.

44. A pharmaceutical dosage form according to claim 43 wherein said dosage form is a tablet.

45. A pharmaceutical dosage form according to claim 43 wherein said dosage form is a capsule.

46. A pharmaceutical dosage form comprising baclofen and a pharmaceutically acceptable excipient, wherein upon oral administration of said pharmaceutical dosage form, the median time period at which at least 80% of said baclofen is absorbed, in vivo, under fasting conditions, is greater than 2.5 hours.

47. A pharmaceutical dosage form according to claim 46 wherein, upon oral administration of said pharmaceutical dosage form, the median time period at which at least 80% of said baclofen is absorbed, in vivo, under fasting conditions, is from about 3 hours to about 4.5 hours.

48. A pharmaceutical dosage form according to claim 47 comprising an enteric-coated controlled release component and an immediate release component.

49. A pharmaceutical dosage form according to claim 48 wherein said enteric-coated controlled release component comprises beads containing a core comprising baclofen, coated with a first, inner polymer layer and a second, outer polymer layer, wherein said outer polymer layer comprises a pH-sensitive polymer.

50. A pharmaceutical dosage form according to claim 48 wherein said enteric-coated controlled release component comprises a polymer selected from the group consisting of: cellulose acetate phthalate, cellulose acetate trimellitate, hydroxypropyl methylcellulose phthalate, polyvinyl acetate phthalate, carboxymethylethylcellulose, co-polymerized methacrylic acid, methacrylic acid methyl esters, and mixtures thereof

51. A pharmaceutical dosage form according to claim 48 wherein, upon oral administration of said dosage form under fasting conditions, said dosage form exhibits an in vivo plasma profile comprising a mean maximum baclofen level from about 2.5 hours to about 5.5 hours after administration.

52. A pharmaceutical dosage form according to claim 48 wherein said dosage form provides a steady-state in vivo plasma profile exhibiting a C_{MIN} at about 12 hours after administration of said dosage form.

53. A pharmaceutical dosage form according claim 46 wherein said baclofen is in the amount from about 2 mg to about 150 mg.

54. A pharmaceutical dosage form according to claim 53 wherein said baclofen is in the amount of about 20 mg.

55. A pharmaceutical dosage form according to claim 53 wherein said baclofen is in the amount of about 25 mg.

56. A pharmaceutical dosage form according to claim 53 wherein said baclofen is in the amount of about 30 mg.

57. A pharmaceutical dosage form according to claim 53 wherein said baclofen is in the amount of about 35 mg.

58. A pharmaceutical dosage form according to claim 53 wherein said baclofen is in the amount of about 40 mg.

59. A pharmaceutical dosage form according to claim 46 wherein the baclofen is formulated as a combination of immediate-release beads and controlled-release beads.

60. A pharmaceutical dosage form according to claim 59 wherein said dosage form is a tablet.

61. A pharmaceutical dosage form according to claim 59 wherein said dosage form is a capsule.

62. A pharmaceutical dosage form comprising baclofen in an immediate release component and in an enteric-coated controlled release component,
wherein said enteric-coated controlled release component comprises a polymer selected from the group consisting of: cellulose acetate phthalate, cellulose acetate trimellitate, hydroxypropyl methylcellulose phthalate, polyvinyl acetate phthalate, carboxymethylethylcellulose, co-polymerized methacrylic acid, methacrylic acid methyl esters, and mixtures thereof; and
wherein upon oral administration of said pharmaceutical dosage form, the median time period at which at least 80% of said baclofen is absorbed, in vivo, under fasting conditions, is from about 3 hours to about 4.5 hours.

63. A pharmaceutical dosage form according to claim 62 wherein said polymer is co-polymerized methacrylic acid.

64. A pharmaceutical dosage form comprising baclofen in an immediate release component and in a controlled release component,
wherein said controlled release component comprises a matrix dosage form; and
wherein upon oral administration of said pharmaceutical dosage form, the median time period at which at least 80% of said baclofen is absorbed, in vivo, under fasting conditions, is from about 3 hours to about 4.5 hours.
